(19) United States
(12) Patent Application Publication
(10) Pub. No.: US 2005/0004056 A1
(43) Pub. Date: Jan. 6, 2005

(54) Ptp10d, tec protein tyrosine kinase and edtp homologous proteins involved in the regulation of energy homeostasis

(76) Inventors: Martin Meise, Hemer (DE); Karsten Eulenberg, Bovenden (DE); Rudiger Fritsch, Gottingen (DE); Thomas Hader, Gottingen (DE); Gunter Bronner, Gottingen (DE); Arnd Steuernagel, Gottingen (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/497,692

(22) PCT Filed: Dec. 4, 2002
(86) PCT No.: PCT/EP02/13744

(30) Foreign Application Priority Data
Dec. 4, 2001 (EP) 01 128 844.6
Dec. 7, 2001 (EP) 01 129 138.2
Jan. 2, 2002 (EP) 01 000 010.5

(51) Int. Cl. A61K 48/00

(52) U.S. Cl. 514/44

(57) ABSTRACT

The present invention discloses PTP10D, Tec protein tyrosine kinase, or EDTP homologous proteins regulating the energy homeostasis and the metabolism of triglycerides, and polynucleotides, which identify and encode the proteins disclosed in this invention. The invention also relates to the use of these sequences in the diagnosis, study, prevention, and treatment of diseases and disorders, for example, but not limited to, metabolic diseases such as obesity as well as related disorders such as eating disorder, cachexia, diabetes mellitus, hypertension, coronary heart disease, hypercholesterolemia, dyslipidemia, osteoarthritis, gallstones, cancers, e.g. cancers of the reproductive organs, and sleep apnea.

This invention relates to the use of nucleic acid sequences encoding PTP10D, Tec protein tyrosine kinase, or EDTP homologous proteins, and the polypeptides encoded thereby and to the use thereof and to the use of effectors thereof in the diagnosis, study, prevention, and treatment of diseases and disorders related to body-weight regulation, for example, but not limited to, metabolic diseases such as obesity as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea.

There are several metabolic diseases of human and animal metabolism, eg., obesity and severe weight loss, that relate to energy imbalance where caloric intake versus energy expenditure is imbalanced. Obesity is one of the most prevalent metabolic disorders in the world. It is a still poorly understood human disease that becomes more and more relevant for western society. Obesity is defined as a body weight more than 20% in excess of the ideal body weight, frequently resulting in a significant impairment of health. Further parameters for defining obesity are waist circumferences, skinfold thickness and bioimpedance (see, inter alia, Kopelman (1999), loc. cit.). Obesity is associated with an increased risk for cardiovascular disease, hypertension, diabetes, hyperlipidaemia and an increased mortality rate. Besides severe risks of illness, individuals suffering from obesity are often isolated socially.

Human obesity is strongly influenced by environmental and genetic factors, whereby the environmental influence is often a hurdle for the identification of (human) obesity genes. Obesity is influenced by genetic, metabolic, biochemical, psychological, and behavioral factors. As such, it is a complex disorder that must be addressed on several fronts to achieve lasting positive clinical outcome. Obese individuals are prone to ailments including: diabetes mellitus, hypertension, coronary heart disease, hypercholesterolemia, dyslipidemia, osteoarthritis, gallstones, cancers of the reproductive organs, and sleep apnea.

Obesity is not to be considered as a single disorder but a heterogeneous group of conditions with (potential) multiple causes. Obesity is also characterized by elevated fasting plasma insulin and an exaggerated insulin response to oral glucose intake (Koltermann, J. Clin. Invest 65, 1980, 1272-1284) and a clear involvement of obesity in type 2 diabetes mellitus can be confirmed (Kopelman, Nature 404, 2000, 635-643).

Hyperlipidemia and elevation of free fatty acids correlate clearly with the “Metabolic Syndrome”, which is defined as the linkage between several diseases, including obesity an insulin resistance. This often occurs in the same patients and are major risk factors for development of Type 2 diabetes and cardiovascular disease. It was suggested that the control of lipid levels and glucose levels is required to treat Type 2 Diabetes, heart disease, and other occurances of Metabolic Syndrome (see, for example, Santomauro A. T. et al., (1999) Diabetes, 48(9):1836-1841).

Even if several candidate genes have been described which are supposed to influence the homeostatic system(s) that regulate body mass/weight, like leptin, VCPI, VCPL, or the peroxisome proliferator-activated receptor-gamma co-activator, the distinct molecular mechanisms and/or molecules influencing obesity or body weight/body mass regulations are not known.

Therefore, the technical problem underlying the present invention was to provide for means and methods for modulating (pathological) metabolic conditions influencing body-weight regulation and/or energy homeostatic circuits. The solution to said technical problem is achieved by providing the embodiments characterized in the claims.

Accordingly, the present invention relates to genes with novel functions in body-weight regulation, energy homeostasis, metabolism, and obesity. The present invention discloses specific genes involved in the regulation of body-weight, energy homeostasis, metabolism, and obesity, and thus in disorders related thereto such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea. More particularly, the present invention describes the human PTP10D gene, the human Tec gene and the human EDTP gene as being involved in those conditions mentioned above.

The term “polynucleotide comprising the nucleotide sequence as shown in GenBank Accession number” relates to the expressible gene of the nucleotide sequences deposited under the corresponding GenBank Accession number. The term “GenBank Accession Number” relates to NCBI GenBank database entries (Ref.: Benson et al., (2000) Nucleic Acids Res. 28: 15-18).

Kinases and phosphatases regulate many different cell proliferation, differentiation, and signaling processes by adding and removing phosphate groups to and from proteins. Reversible protein phosphorylation is the main strategy for controlling activities of eukaryotic cells. It is estimated that more than 1000 of the 10000 proteins active in a typical mammalian cell are phosphorylated. The high energy phosphate which drives activation is generally transferred from adenosine triphosphate molecules (ATP) to a particular protein by protein kinases and removed from that protein by protein phosphatases. Phosphorylation occurs in response to extracellular signals (hormones, neurotransmitters, growth and differentiation factors, etc.), cell cycle checkpoints, and environmental or nutritional stresses and is roughly analogous to turning on a molecular switch. When the switch goes on, the appropriate protein kinase activates a metabolic enzyme, regulatory protein, receptor, cytoskeletal protein, ion channel or pump, or transcription factor. Uncontrolled signaling has been implicated in a variety of disease conditions including inflammation, cancer, arteriosclerosis, and psoriasis.

Tyrosine Phosphatases:

Protein tyrosine phosphorylation has been shown to be an evolutionary conserved mechanism essential for the regulation of numerous cellular events, including cell growth, tissue differentiation and metabolism (Walton K. M. and Dixon J. E., (1993) Annu Rev Biochem 62:101-120; Li L. and Dixon J. E., (2000) Semin Immunol. 12(1):75-84). Several hundred protein tyrosine phosphatases (herein referred to as PTP) are encoded by the human genome that are subdivided into different classes. Based on subcellular localisation PTPs are classified as receptor-like or intracellular. Receptor-like PTPs contain a transmembrane domain, a receptor-like extracellular domain, and usually two intracellular PTP domains. Only few receptor PTPs contain only one intracellular PTP domain (e.g. Drosophila PTP10D and human PTPRB). Characteristics of the extracellular domain are used to further subdivide receptor PTPs into five different types (Mourey R. J. and Dixon, J. E., (1994) Curr Opin Genet Dev 4(1):31-39). Drosophila PTP10D, for example, contains a series of extracellular fibronectin-like repeats and immunoglobulin-like repeats and belongs to Type III receptor PTPs. The only known function of Drosophila PTP10D is an involvement in the control of axon guidance during embryonic development (Sun Q. et al., (2000) Development 127:801-812; Kraut R. et al., (2001) Curr Biol 11:417-430). No function in the regulation of metabolism has been reported for PTP10D or its human homolog.

EDTP:

Drosophila gene CG6542 encodes for the egg-derived tyrosine phosphatase (EDTP) that shows a tyr-phosphatase-1 protein domain characteristic for the tyrosine specific protein phosphatase and dual specificity protein phosphatase family. EDTP, which shows tyrosine phoshatase activity, participates in the embryogenesis of Sarcophaga peregrina (flesh fly) (see, Yamaguchi S. et al., (1999) Eur J Biochem 259(3):946-953). No further functions were described for this protein in the prior art.

Tec:

The Tec family is a recently emerging subfamily of non-receptor protein-tyrosine kinases (PTKs) represented by its first member, Tec. This family is composed of five members, namely Tec, Btk, Itk/Emt/Tsk, Bmx and Txk/Rlk (see, for example, Smith C. I. et al., (2001), Bioessays 23(5):436-446). They are characterized by having four structural modules: PH (pleckstrin homology) domain, SH3 (Src homology 3) domain, SH2 (Src homology 2) domain and kinase (Src homology 1) domain. The most characteristic feature of this family is the presence of a PH domain in their protein structure. The PH domain is known to bind phosphoinositides; on this basis, Tec family PTKs may act as merge points of phosphotyrosine-mediated and phospholipid-mediated signaling systems.

Many Tec family proteins are abundantly expressed in hematopoietic tissues, and are presumed to play important roles in the growth and differentiation processes of blood cells. Supporting this, mutations in the Btk gene cause X chromosome-linked agammaglobulinemia (XLA) in humans and X chromosome-linked immunodeficiency (Xid) in mice, indicating that Btk activity is indispensable for B-cell ontogeny central but diverse modulatory roles in various cellular processes. Members of the human Tec kinase family play an important role in signaling through antigen-receptors such as the T-cell antigen receptor (TCR), B-cell antigen receptor (BCR) and Fc-epsilon receptor. Recent studies have generated new insights into the domains in Tec kinases that take part in intramolecular and intermolecular binding. Genetic studies of kinase-knockout mice have emphasized the importance of Tec kinases in lymphocyte development, differentiation and apoptosis.

Tec kinases participate in signal transduction in response to virtually all types of extracellular stimuli which are transmitted by growth factor receptors, cytokine receptors, G-protein coupled receptors, antigen-receptors and integrins (see for example, Lewis C. M. et al, (2001), Curr. Opin. Immunol. 13(3):317-325; Cantrell D. A., (2001), J. Cell Sci. 114(Pt 8):1439-1445; Qiu Y. and Kung H. J., (2000), Oncogene 19(49):5651-5661). The kinases have also a function in cytoskeletal reorganization and cell motility (see, for example, Tsoukas C. D. et al., (2001), Trends Immunol. 22(1):17-20).

Drosophila Btk29A is most homologous to mammalian non-receptor type protein-tyrosine kinases, such as human tec protein tyrosine kinase and mouse protein tyrosine kinase TecIV. The human Tec protein is highly homologous to those of the members of the Tec family including mouse Tec type IV (94% homology), mouse Tsk/Itk (60%), and human Btk (57%) (Sato K. et al., (1994), Leukemia 8(10):1663-1672). The homology between human Tec and other members of the Tec family can be observed not only in the Src homology 3 (SH3), SH2, and kinase domains, but also in the N-terminal unique domain. Mouse TecIV is highly expressed in many hematopoietic cell lines.

So far, it has not been described that PTP10D, Btk29A, or EDTP, or the human homologous proteins are involved in the regulation of energy homeostasis and body-weight regulation and related disorders, and thus, no functions in metabolic diseases and other diseases as listed above have been discussed. In this invention we demonstrate that the correct gene dose of PTP10D, Tec protein tyrosine kinase, or EDTP is essential for maintenance of energy homeostasis. A genetic screen was used to identify that a mutation of a PTP10D, Tec, or EDTP homologous gene causes obesity, reflected by a significant increase of triglyceride content, the major energy storage substance.

Polynucleotides encoding a protein with homologies to PTP10D, Tec, or EDTP are suitable to investigate diseases and disorders as described above. Further new compositions useful in diagnosis, treatment, and prognosis of diseases and disorders as described above are provided.

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure.

The present invention discloses that PTP10D, Tec, or EDTP homologous proteins are regulating the energy homeostasis and fat metabolism especially the metabolism and storage of triglycerides, and polynucleotides, which identify and encode the proteins disclosed in this invention. The invention also relates to vectors, host cells, antibodies, and recombinant methods for producing the polypeptides and polynucleotides of the invention. The invention also relates to the use of these sequences in the diagnosis, study, prevention, and treatment of diseases and disorders, for example, but not limited to, metabolic diseases such as obesity as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea.

PTP10D, Tec, or EDTP homologous proteins and nucleic acid molecules coding therefore are obtainable from insect or vertebrate species, e.g. mammals or birds. Particularly preferred are homologous nucleic acids, particularly nucleic acids encoding a human RPTP beta protein, a mouse PTP10D like protein, a human tec protein tyrosine kinase protein and mouse protein tyrosine kinase TecIV or a human or mouse EDTP protein.

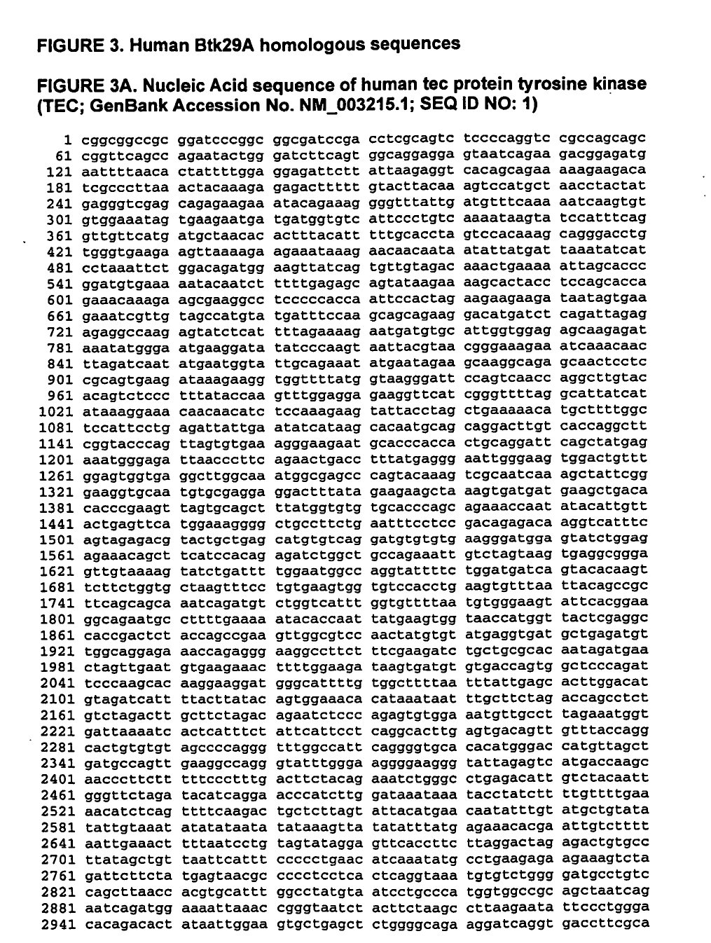

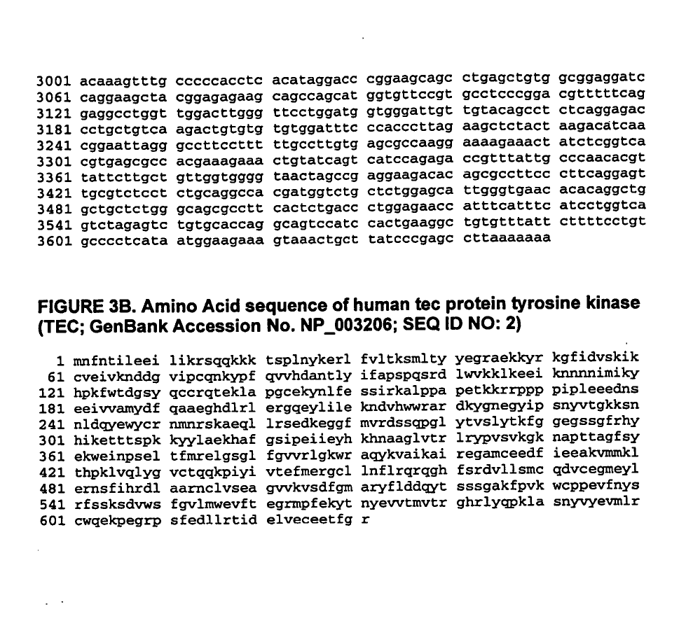

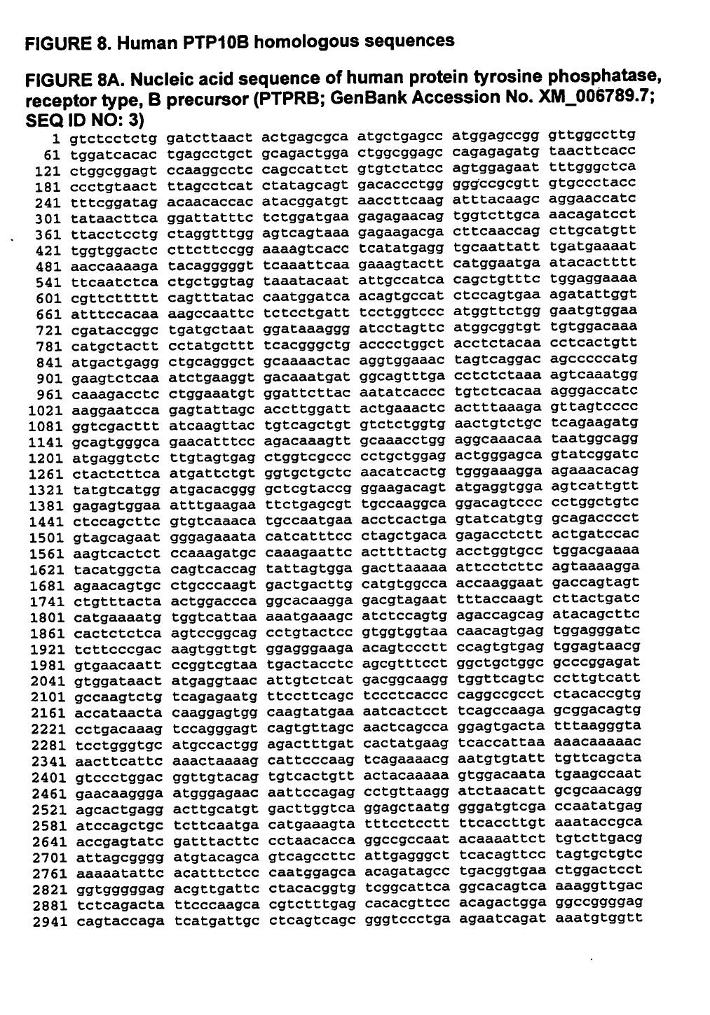

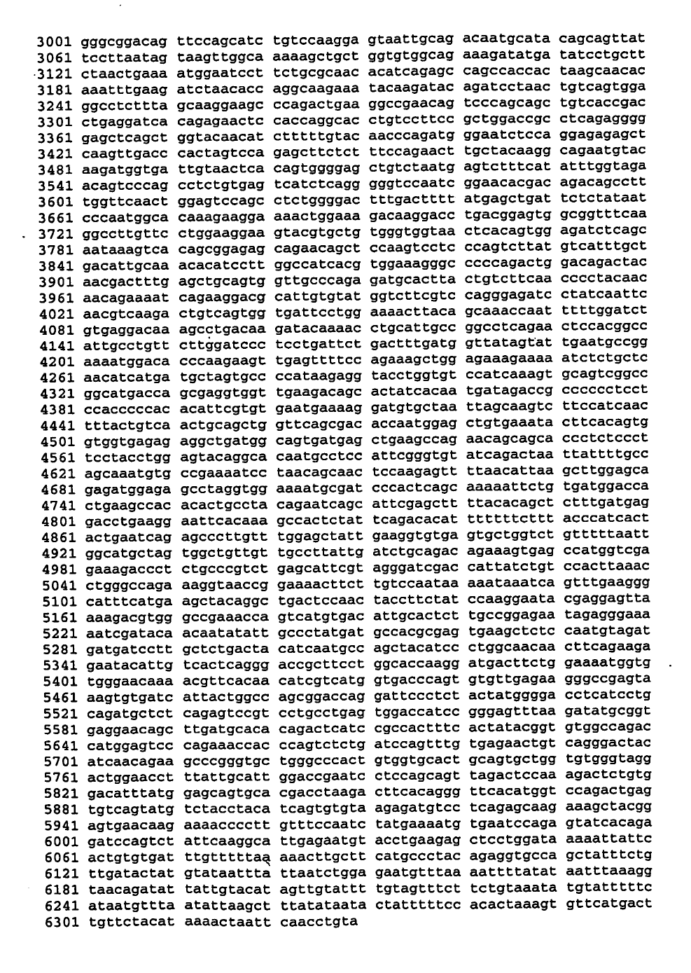

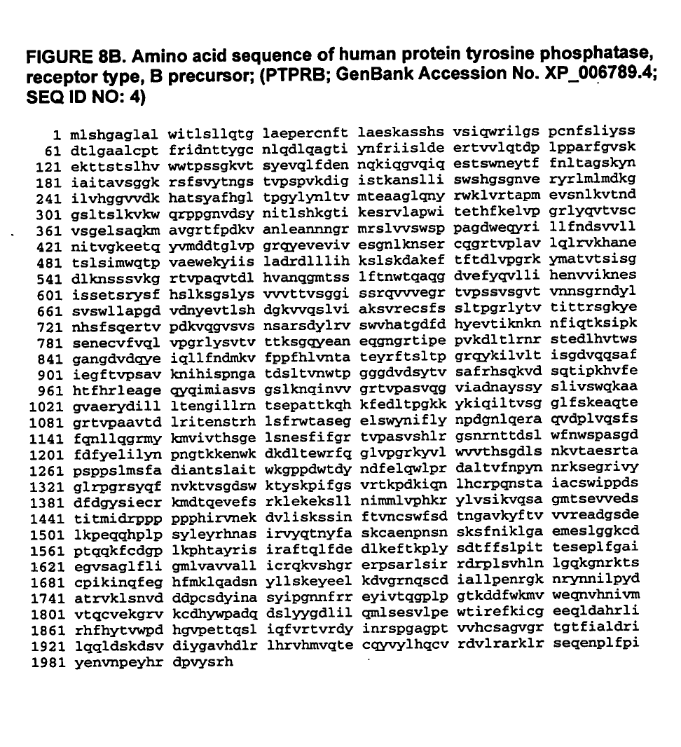

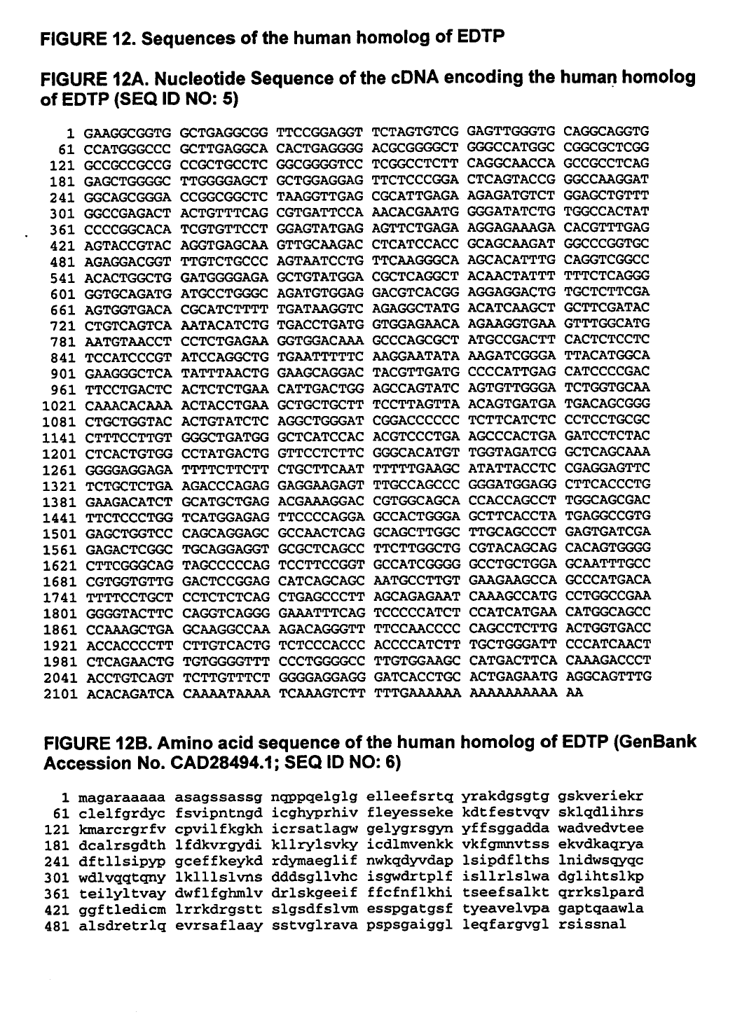

In this invention we particularly refer to protein tyrosin phosphatase 10D (referred to as PTP10D), Bruton's tyrosine kinase 29A (referred to as Btk29A), and Egg-derived tyrosine phosphatase (referred to as EDTP), and PTP10D, Btk29A, and EDTP homologous proteins (for example protein tyrosine phosphatase, receptor type, Beta (PTPRB or RPTP beta), tec protein tyrosin kinase (Tec), mammalian EDTP homologous proteins), which include Drosophila and mammalian, preferably human, homologous polypeptides or proteins or sequences encoding those proteins as proteins of the invention. Especially preferred embodiments are:
Drosophila PTP10D (Gadfly Accession Number CG1817), human PTPRB (Genbank Accession Number XM_{—}006789.7 for the cDNA (SEQ ID NO: 3, see FIG. 8A), Genbank Accession Number XP_{—}006789.4 for the protein (SEQ ID NO: 4, see FIG. 8B), and mouse receptor-type protein tyrosine phosphatase (Genbank Accession Number AF157628 for the cDNA, Genbank Accession Number AAF80346 for the protein),
Drosophila Btk29A (Gadfly Accession Numbers CG18533, CG8049), human tec protein tyrosine kinase (Genbank Accession Number NM_{—}003215.1 (formerly XM_{—}044444.3) for the cDNA (SEQ ID NO: 1, see FIG. 3A), Genbank Accession Number NP_{—}003206.1 (formerly XP_{—}044444) for the protein (SEQ ID NO: 2, see FIG. 3B), and mouse protein tyrosine kinase TecIV (Genbank Accession Number AAD43402.1)
Drosophila EDTP (Gadfly Accession Number CG6542), human EDTP homologous proteins on chromosome 3 ((Genbank Accession Numbers XM_{—}056609.3 and NM_{—}022485.2 for the cDNAs, XP_{—}056609 and NP_{—}071930 for the proteins), cDNA of SEQ ID NO: 5 (FIG. 12A) encoding protein of SEQ ID NO: 6 (FIG. 12B), predicted cDNA from Genbank Accession Numbers, XM_{—}056748, and BC_{—}001674, further human proteins with Genbank Accession Numbers AAH35690.1, AAL55787, BAC11211, and CAD28494).

The invention particularly relates to a nucleic acid molecule encoding a polypeptide contributing to regulating the energy homeostasis and the metabolism of triglycerides, wherein said nucleic acid molecule comprises
(a) the nucleotide sequence encoding a protein of the invention and/or a sequence complementary thereto,
(b) a nucleotide sequence which hybridizes at 50° C. in a solution containing 1×SSC and 0.1% SDS to a sequence of (a),
(c) a sequence corresponding to the sequences of (a) or (b) within the degeneration of the genetic code,
(d) a sequence which encodes a polypeptide which is at least 85%, preferably at least 90%, more preferably at least 95%, more preferably at least 98% and up to 99.6% identical to the amino acid sequence of a protein of the invention,
(e) a sequence which differs from the nucleic acid molecule of (a) to (d) by mutation and wherein said mutation causes an alteration, deletion, duplication and/or premature stop in the encoded polypeptide or
(f) a partial sequence of any of the nucleotide sequences of (a) to (e) having a length of at least 15 bases, preferably at least 20 bases, more preferably at least 25 bases and most preferably at least 50 bases.

The invention is based on the finding that the proteins of the invention and the polynucleotides encoding these, are involved in the regulation of triglyceride storage and therefore energy homeostasis. The invention describes the use of these proteins and polynucleotides or fragments thereof or effectors thereof, e.g. antibodies, aptamers, antisense molecules, RNAi molecules, ribozymes, peptides or low-molecular weight organic compounds or other receptors recognizing the polynucleotide or the polypeptide for the diagnosis, study, prevention, or treatment of diseases and disorders related thereto, including metabolic diseases such as obesity as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea.

Accordingly, the present invention relates to genes with novel functions in body-weight regulation, energy homeostasis, metabolism, and obesity. To find genes with novel functions in energy homeostasis, metabolism, and obesity, a functional genetic screen was performed with the model organism Drosophila melanogaster (Meigen). Drosophila melanogaster is one of the most intensively studied organisms in biology and serves as a model system for the investigation of many developmental and cellular processes common to higher eukaryotes, including humans (see, for example, Adams et al., (2000) Science 287: 2185-2195). The success of Drosophila melanogaster as a model organism is largely due to the power of forward genetic screens to identify the genes that are involved in a biological process (see, Johnston (2002) Nat Rev Genet 3: 176-188; Rorth, (1996) Proc Natl Acad Sci USA 93: 12418-12422). One resource for screening was a proprietary Drosophila melanogaster stock collection of EP-lines. The P-vector of this collection has Gal4-UAS-binding sites fused to a basal promoter that can transcribe adjacent genomic Drosophila sequences upon binding of Gal4 to UAS-sites. This enables the EP-line collection for overexpression of endogenous flanking gene sequences. In addition, without activation of the UAS-sites, integration of the EP-element into the gene is likely to cause a reduction of gene activity, and allows determining its function by evaluating the loss-of-function phenotype.

Triglycerides are the most efficient storage for energy in cells, and are significantly increased in obese patients. In this invention, we have used a genetic screen to identify, that mutations of genes encoding the proteins of the invention or homologous genes cause changes in the body weight which is reflected by a significant change in the triglyceride levels. In order to isolate genes with a function in energy homeostasis, several thousand proprietary and publicly available EP-lines were tested for their triglyceride content after a prolonged feeding period (illustrated in more detail in the EXAMPLES). Lines with significantly changed triglyceride content were selected as positive candidates for further analysis.

In this invention, the content of triglycerides of a pool of flies with the same genotype after feeding for six days was analyzed using a triglyceride assay, as, for example, but not for limiting the scope of the invention, is described in more detail below in the examples section. The change of triglyceride content due to the loss of a gene function suggests gene activities in energy homeostasis in a dose dependent manner that controls the amount of energy stored as triglycerides.

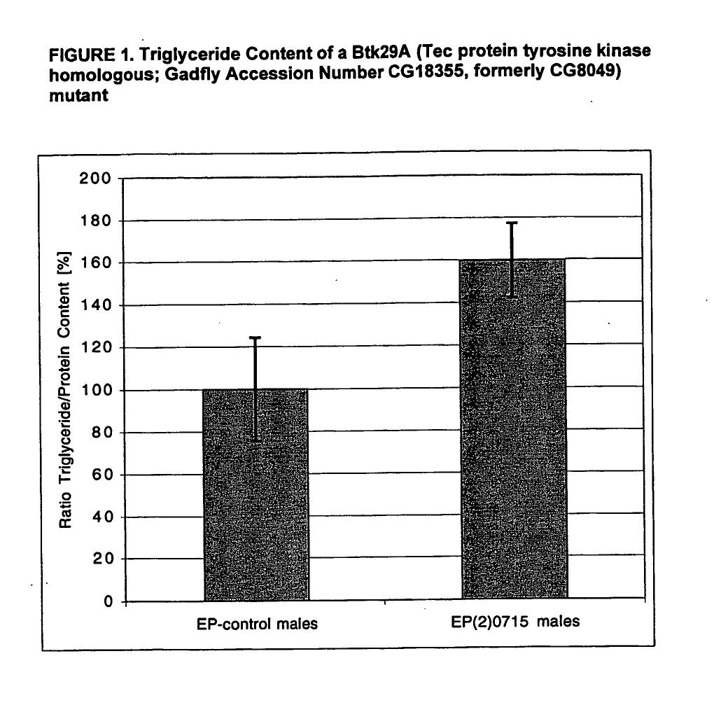

The result of the triglyceride content analysis is shown in FIGS. 1, 5, and 10. We found that homozygous EP(2)0715, EP(2)2389, and EP(2)2390 flies, hemizygous PX7194.1 flies and heterozygous EP(2)2389, and EP(2)2390 flies have a higher triglyceride content than the controls (average triglyceride levels). Therefore, the loss of a gene activity in the gene loci, where the EP-vectors are integrated, is responsible for changes in the metabolism of the energy storage triglycerides, therefore representing in all cases an obese fly model. The increase of triglyceride content due to the loss of a gene function suggests gene activities in energy homeostasis in a dose dependent manner that controls the amount of energy stored as triglycerides.

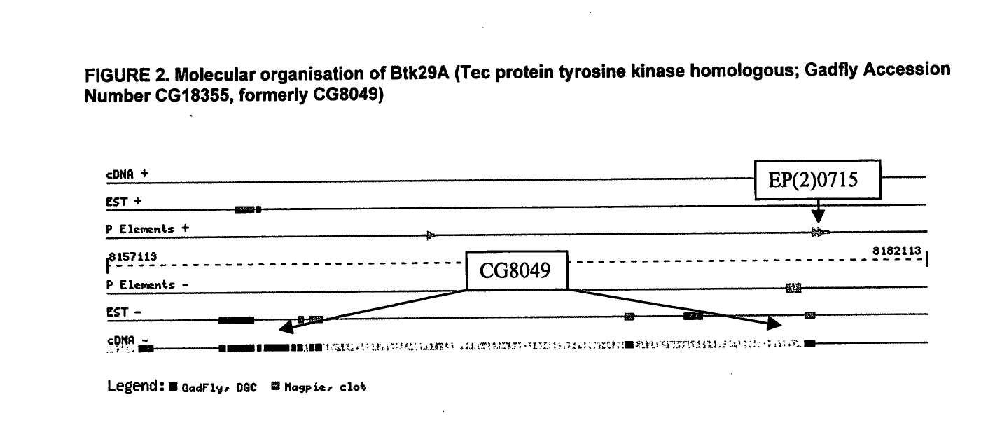

Nucleic acids encoding the proteins of the present invention were identified using a iPCR technique. Genomic DNA sequences were isolated that are localised directly 3′ to the EP vectors (herein EP(2)0715, EP(2)2389, and EP(2)2390) integration and 5′ to the EP vectors (herein PX7194.1 integration). Using those isolated genomic sequences public databases like Berkeley Drosophila Genome Project (GadFly, see also FlyBase (1999) Nucleic Acids Research 27:85-88) were screened thereby confirming the homozygous or hemizygous viable or heterozygous integration site of the vectors into the transcription units of the genes FIGS. 2, 6, and 11 show the molecular organisation of these gene loci.

The proteins of the invention and homologous proteins and nucleic acid molecules coding therefore are obtainable from insect or vertebrate species, e.g. mammals or birds. Particularly preferred are nucleic acids encoding the human homologs of the proteins of the invention. The present invention is describing polypeptides comprising the amino acid sequences of the proteins of the invention. Comparisons (ClustalX 1.8 analysis or ClustalW 1.82 analysis, see for example Thompson J. D. et al., (1994) Nucleic Acids Res. 22(22):4673-4680; Thompson J. D., (1997) Nucleic Acids Res 25(24):4876-4882; Higgins, D. G. et al., (1996) Methods Enzymol. 266:383-402) between the respective proteins of different species (human and Drosophila) were conducted. Based upon homology, the Drosophila proteins of the invention and each homologous protein or peptide may share at least some activity. No functional data described the regulation of body weight control and related metabolic diseases such as obesity are available in the prior art for the genes claimed in this invention.

Expression profiling studies (see Examples for more detail) confirm the particular relevance of the proteins, e.g. PTPRP and Tec kinase as regulators of energy metabolism in mammals.

The invention also encompasses polynucleotides that encode the proteins of the invention. Accordingly, any nucleic acid sequence, which encodes the amino acid sequences of a protein of the invention can be used to generate recombinant molecules that express a protein of the invention. In a particular embodiment, the invention encompasses the polynucleotide encoding a Drosophila or human protein of the invention. It will be appreciated by those skilled in the art that as a result of the degeneracy of the genetic code, a multitude of nucleotide sequences encoding the proteins of the invention, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequences of naturally occurring proteins of the invention, and all such variations are to be considered as being specifically disclosed. Although nucleotide sequences which encode the proteins of the invention and its variants are preferably capable of hybridising to the nucleotide sequences of the naturally occurring proteins of the invention under appropriately selected conditions of stringency, it may be advantageous to produce nucleotide sequences encoding the proteins of the invention or their derivatives possessing a substantially different codon usage. Codons may be selected to increase the rate at which expression of the peptide occurs in a particular prokaryotic or eukaryotic host in accordance with the frequency with which particular codons are utilised by the host. Other reasons for substantially altering the nucleotide sequence encoding a protein of the invention and its derivatives without altering the encoded amino acid sequences include the production of RNA transcripts having more desirable properties, such as a greater half-life, than transcripts produced from the naturally occurring sequences. The invention also encompasses production of DNA sequences, or portions thereof, which encode the proteins of the invention and their derivatives, entirely by synthetic chemistry. After production, the synthetic sequence may be inserted into any of the many available expression vectors and cell systems using reagents that are well known in the art at the time of the filing of this application. Moreover, synthetic chemistry may be used to introduce mutations into a sequence encoding a protein of the invention or any portion thereof.

Also encompassed by the invention are polynucleotide sequences that are capable of hybridising to the claimed nucleotide sequences, and in particular, those of the polynucleotide encoding a protein of the invention under various conditions of stringency. Hybridisation conditions are based on the melting temperature (Tm) of the nucleic acid binding complex or probe, as taught in Wahl, G. M. and S. L. Berger (1987: Methods Enzymol. 152:399-407) and Kimmel, A. R. (1987; Methods Enzymol. 152:507-511), and may be used at a defined stringency. Preferably, hybridization under stringent conditions means that after washing for 1 h with 1×SSC and 0.1% SDS at 50° C., preferably at 55° C., more preferably at 62° C. and most preferably at 68° C., particularly for 1 h in 0.2×SSC and 0.1% SDS at 50° C., preferably at 55° C., more preferably at 62° C. and most preferably at 68° C., a positive hybridization signal is observed. Altered nucleic acid sequences encoding the proteins of the invention which are encompassed by the invention include deletions, insertions, or substitutions of different nucleotides resulting in a polynucleotide that encodes the same or a functionally equivalent protein of the invention.

The encoded proteins may also contain deletions, insertions, or substitutions of amino acid residues, which produce a silent change and result in a functionally equivalent protein of the invention. Deliberate amino acid substitutions may be made on the basis of similarity in polarity, charge, solubility, hydrophobicity, hydrophilicity, and/or the amphipathic nature of the residues as long as the biological activity of the proteins of the invention is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; phenylalanine and tyrosine. Furthermore, the invention relates to peptide fragments of the proteins or derivatives thereof such as cyclic peptides, retro-inverso-peptides or peptide mimetics having a length of at least 4, preferably at least 6 and up to 50 amino acids.

Also included within the scope of the present invention are alleles of the genes encoding the proteins of the invention. As used herein, an “allele” or “allelic sequence” is an alternative form of the gene, which may result from at least one mutation in the nucleic acid sequence. Alleles may result in altered mRNAs or polypeptides whose structures or function may or may not be altered. Any given gene may have none, one, or many allelic forms. Common mutational changes, which give rise to alleles, are generally ascribed to natural deletions, additions, or substitutions of nucleotides. Each of these types of changes may occur alone, or in combination with the others, one or more times in a given sequence.

The nucleic acid sequences encoding the proteins of the invention may be extended utilising a partial nucleotide sequence and employing various methods known in the art to detect upstream sequences such as promoters and regulatory elements. For example, one method which may be employed, “restriction-site” PCR, uses universal primers to retrieve unknown sequence adjacent to a known locus (Sarkar, G. (1993) PCR Methods Applic. 2:318-322). Inverse PCR may also be used to amplify or extend sequences using divergent primers based on a known region (Triglia, T. et al. (1988) Nucleic Acids Res. 16:8186).

Another method which may be used is capture PCR which involves PCR amplification of DNA fragments adjacent to a known sequence in human and yeast artificial chromosome DNA (Lagerstrom, M. et al. (PCR Methods Applic. 1:111-119). Another method which may be used to retrieve unknown sequences is that of Parker, J. D. et al. (1991; Nucleic Acids Res. 19:3055-3060). Additionally, one may use PCR, nested primers, and PROMOTERFINDER libraries to walk in genomic DNA (Clontech, Palo Alto, Calif.). This process avoids the need to screen libraries and is useful in finding intron/exon junctions.

In order to express biologically active proteins of the invention, the nucleotide sequences encoding the proteins of the invention or functional equivalents, optionally in the form of fusion proteins, may be inserted into appropriate expression vectors, i.e., a vector, which contains the necessary elements for the transcription and translation of the inserted coding sequence. Methods, which are well known to those skilled in the art, may be used to construct expression vectors containing sequences encoding a protein of the invention and appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

Regulatory elements include for example a promoter, an initiation codon, a stop codon, a mRNA stability regulatory element, and a polyadenylation signal. Expression of a polynucleotide can be assured by (i) constitutive promoters such as the Cytomegalovirus (CMV) promoter/enhancer region, (ii) tissue specific promoters such as the insulin promoter (see, Soria et al., 2000, Diabetes 49:157), SOX2 gene promotor (see Li et al., 1998, Curr. Biol. 8:971-4), Msi-1 promotor (see Sakakibara et al., 1997, J. Neuroscience 17:8300-8312), alpha-cardia myosin heavy chain promotor or human atrial natriuretic factor promotor (Klug et al., 1996, J. clin. Invest 98:216-24; Wu et al., 1989, J. Biol. Chem. 264:6472-79) or (iii) inducible promoters such as the tetracycline inducible system. Expression vectors can also contain a selection agent or marker gene that confers antibiotic resistance such as the neomycin, hygromycin or puromycin resistance genes. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y. and Ausubel, F. M. et al. (1989) Current Protocols in Molecular Biology, John Wiley & Sons, New York, N.Y. In a further embodiment of the invention, natural, modified or recombinant nucleic acid sequences encoding the proteins of the invention and homologous proteins may be ligated to a heterologous sequence to encode a fusion protein.

A variety of expression vector/host systems may be utilised to contain and express sequences encoding the proteins of the invention. These include, but are not limited to, micro-organisms such as bacteria transformed with recombinant bacteriophage, plasmid, or cosmid DNA expression vectors; yeast transformed with yeast expression vectors; insect cell systems infected with virus expression vectors (e.g., baculovirus, adenovirus, adeno-associated virus, lentivirus, retrovirus); plant cell systems transformed with virus expression vectors (e.g., cauliflower mosaic virus, CaMV; tobacco mosaic virus, TMV) or with bacterial expression vectors (e.g., Ti or PBR322 plasmids); or animal cell systems.

The presence of polynucleotide sequences encoding the proteins of the invention can be detected by DNA-DNA or DNA-RNA hybridisation and/or amplification using probes or portions or fragments of polynucleotides encoding a protein of the invention. Nucleic acid amplification based assays involve the use of oligonucleotides or oligomers based on the sequences encoding said protein of the invention to detect transformants containing DNA or RNA encoding said protein of the invention. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides, which can be used as a probe or amplimer.

A variety of protocols for detecting and measuring the expression of the proteins of the invention, using either polyclonal or monoclonal antibodies specific are known in the art. Examples include enzyme-linked immunosorbent assay (ELISA), radioimmunoassay (RIA), and fluorescence activated cell sorting (FACS). A two-site, monoclonal-based immunoassay utilising monoclonal antibodies reactive to two non-interfering epitopes on the proteins of the invention is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labelled hybridisation or PCR probes for detecting sequences related to polynucleotides encoding a protein of the invention include oligo-labelling, nick translation, end-labelling or PCR amplification using a labelled nucleotide, or enzymatic synthesis. These procedures may be conducted using a variety of commercially available kits (Pharmacia & Upjohn, (Kalamazoo, Mich.); Promega (Madison Wis.); and U.S. Biochemical Corp., (Cleveland, Ohio).

Suitable reporter molecules or labels, which may be used, include radionuclides, enzymes, fluorescent, chemiluminescent, or chromogenic agents as well as substrates, co-factors, inhibitors, magnetic particles, and the like.

Host cells transformed with nucleotide sequences encoding the proteins of the invention may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides which encode the proteins of the invention may be designed to contain signal sequences, which direct secretion of the proteins of the invention through a prokaryotic or eukaryotic cell membrane. Other recombinant constructions may be used to join sequences encoding the proteins of the invention to nucleotide sequence encoding a polypeptide domain, which will facilitate purification of soluble proteins. Such purification facilitating domains include, but are not limited to, metal chelating peptides such as histidine-tryptophan modules that allow purification on immobilised metals, protein A domains that allow purification on immobilised immunoglobulin, and the domain utilised in the FLAG extension/affinity purification system (Immunex Corp., Seattle, Wash.) The inclusion of cleavable linker sequences such as those specific for Factor XA or Enterokinase (Invitrogen, San Diego, Calif.) between the purification domain and the proteins of the invention may be used to facilitate purification. One such expression vector provides for expression of a fusion protein containing the proteins of the invention and a nucleic acid encoding 6 histidine residues preceding a Thioredoxine or an Enterokinase cleavage site. The histidine residues facilitate purification on IMIAC (immobilised metal ion affinity chromatography as described in Porath, J. et al. (1992, Prot. Exp. Purif. 3: 263-281)) while the Enterokinase cleavage site provides a means for purifying the proteins of the invention from the fusion protein. A discussion of vectors which contain fusion proteins is provided in Kroll, D. J. et al. (1993; DNA Cell Biol. 12:441-453). In addition to recombinant production, fragments of the proteins of the invention may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431 A peptide synthesiser (Perkin Elmer). Various fragments of the proteins of the invention may be chemically synthesised separately and combined using chemical methods to produce the full length molecule.

Diagnostics and Therapeutics

The data disclosed in this invention show that the nucleic acids and proteins of the invention and effectors thereof are useful in diagnostic and therapeutic applications implicated, for example but not limited to, in metabolic disorders such as obesity as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea. Hence, diagnostic and therapeutic uses for the nucleic acids and proteins of the invention are, for example but not limited to, the following: (i) protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention are useful in diagnostic and therapeutic applications implicated in various applications as described below. For example, but not limited to, cDNAs encoding the proteins of the invention and particularly their human homologues may be useful in gene therapy, and the proteins of the invention and particularly their human homologues may be useful when administered to a subject in need thereof. By way of non-limiting example, the compositions of the present invention will have efficacy for treatment of patients suffering from, for example, but not limited to, in metabolic disorders as described above.

The nucleic acids encoding the proteins of the invention, or fragments thereof, may further be useful in diagnostic applications, wherein the presence or amount of the nucleic acids or the proteins are to be assessed. These materials are further useful in the generation of antibodies that bind immunospecifically to the substances of the invention for use in therapeutic or diagnostic methods.

For example, in one aspect, antibodies which are specific for a protein of the invention may be used directly as an antagonist, or indirectly as a targeting or delivery mechanism for bringing a pharmaceutical agent to cells or tissue which express said protein of the invention. The antibodies may be generated using methods that are well known in the art. Such antibodies may include, but are not limited to, polyclonal, monoclonal, chimerical, single chain, Fab fragments, and fragments produced by a Fab expression library. Neutralising antibodies, (i.e., those which inhibit dimer formation) are especially preferred for therapeutic use.

For the production of antibodies, various hosts including goats, rabbits, rats, mice, humans, and others, may be immunised by injection with a protein of the invention, or any fragment or oligopeptide thereof which has immunogenic properties. Depending on the host species, various adjuvants may be used to increase immunological response. It is preferred that the peptides, fragments, or oligopeptides used to induce antibodies to a protein of the invention have an amino acid sequence consisting of at least five amino acids, and more preferably at least 10 amino acids.

Monoclonal antibodies to a protein of the invention may be prepared using any technique which provides for the production of antibody molecules by continuous cell lines in culture. These include, but are not limited to, the hybridoma technique, the human B-cell hybridoma technique, and the EBV-hybridoma technique (Köhler, G. et al. (1975) Nature 256:495-497; Kozbor, D. et al. (1985) J. Immunol. Methods 81:31-42; Cote, R. J. et al. Proc. Natl. Acad. Sci. 80:2026-2030; Cole, S. P. et al. (1984) Mol. Cell Biol. 62:109-120).

In addition, techniques developed for the production of “chimeric antibodies”, the splicing of mouse antibody genes to human antibody genes to obtain a molecule with appropriate antigen specificity and biological activity can be used (Morrison, S. L. et al. (1984) Proc. Natl. Acad. Sci. 81:6851-6855; Neuberger, M. S. et al (1984) Nature 312:604-608; Takeda, S. et al. (1985) Nature 314:452-454). Alternatively, techniques described for the production of single chain antibodies may be adapted, using methods known in the art, to produce single chain antibodies specific for a protein of the invention. Antibodies with related specificity, but of distinct idiotypic composition, may be generated by chain shuffling from random combinatorial immunoglobulin libraries (Burton, D. R. (1991) Proc. Natl. Acad. Sci. 88:11120-3). Antibodies may also be produced by inducing in vivo production in the lymphocyte population or by screening recombinant immunoglobulin libraries or panels of highly specific binding reagents as disclosed in the literature (Orlandi, R. et al. (1989) Proc. Natl. Acad. Sci. 86:3833-3837; Winter, G. et al. (1991) Nature 349:293-299).

Antibody fragments, which contain specific binding sites for a protein of the invention, may also be generated. For example, such fragments include, but are not limited to, the F(ab′)₂ fragments which can be produced by Pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse, W. D. et al. (1989) Science 254:1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between a protein of the invention and its specific antibody. A two-site, monoclonal-based immunoassay utilising monoclonal antibodies reactive to two non-interfering epitopes of a protein of the invention is preferred, but a competitive binding assay may also be employed (Maddox, supra).

In another embodiment of the invention, the polynucleotides encoding the proteins of the invention, or any fragment thereof, or nucleic acid effector molecules such as antisense molecules, aptamers, RNAi molecules or ribozymes may be used for therapeutic purposes. In one aspect, aptamers, i.e. nucleic acid molecules, which are capable of binding to a protein of the invention and modulating its activity may be generated by a screening and selection procedure involving the use of combinatorial nucleic acid libraries.

In a further aspect, antisense molecules to the polynucleotide encoding a protein of the invention may be used in situations in which it would be desirable to block the transcription of the mRNA. In particular, cells may be transformed with sequences complementary to polynucleotides encoding a protein of the invention. Thus, antisense molecules may be used to modulate the activity of the proteins of the invention, or to achieve regulation of gene function. Such technology is now well know in the art, and sense or antisense oligomers or larger fragments, can be designed from various locations along the coding or control regions of sequences encoding the proteins of the invention. Expression vectors derived from retroviruses, adenovirus, herpes or vaccinia viruses, or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of the gene encoding a protein of the invention. These techniques are described both in Sambrook et al. (supra) and in Ausubel et al. (supra). Genes encoding the proteins of the invention can be turned off by transforming a cell or tissue with expression vectors which express high levels of polynucleotide or fragment thereof which encodes a protein of the invention. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing antisense molecules, DNA, RNA, or PNA, to the control regions of the gene encoding a protein of the invention, i.e., the promoters, enhancers, and introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors, or regulatory molecules. Recent therapeutic advances using triplex DNA have been described in the literature (Gee, J. E. et al. (1994) In; Huber, B. E. and B. I. Carr, Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyse the specific cleavage of RNA. The mechanism of ribozyme action involves sequence-specific hybridisation of the ribozyme molecule to complementary target RNA, followed by endonucleolytic cleavage. Examples, which may be used, include engineered hammerhead motif ribozyme molecules that can be specifically and efficiently catalyse endonucleolytic cleavage of sequences encoding a protein of the invention. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridisation with complementary oligonucleotides using ribonuclease protection assays.

Nucleic acid effector molecules, e.g. antisense molecules and ribozymes of the invention may be prepared by any method known in the art for the synthesis of nucleic acid molecules. These include techniques for chemically synthesising oligonucleotides such as solid phase phosphoramidite chemical synthesis. Alternatively, RNA molecules may be generated by in vitro and in vivo transcription of DNA sequences encoding the proteins of the invention. Such DNA sequences may be incorporated into a variety of vectors with suitable RNA polymerase promoters such as T7 or SP6. Alternatively, these cDNA constructs that synthesise antisense RNA constitutively or inducibly can be introduced into cell lines, cells, or tissues. RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognised by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may consist of a protein of the invention, antibodies to a protein of the invention, mimetics, agonists, antagonists, or inhibitors of a protein of the invention. The compositions may be administered alone or in combination with at least one other agent, such as stabilising compound, which may be administered in any sterile, biocompatible pharmaceutical carrier, including, but not limited to, saline, buffered saline, dextrose, and water. The compositions may be administered to a patient alone, or in combination with other agents, drugs or hormones. The pharmaceutical compositions utilised in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries, which facilitate processing of the active compounds into preparations which, can be used pharmaceutically. Further details on techniques for formulation and administration may be found in the latest edition of Remington's Pharmaceutical Sciences (Maack Publishing Co., Easton, Pa.). Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilising processes. After pharmaceutical compositions have been prepared, they can be placed in an appropriate container and labelled for treatment of an indicated condition. For administration of the proteins of the invention, such labelling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective does can be estimated initially either in cell culture assays, e.g., of preadipocyte cell lines, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, for example a nucleic acid or protein of the invention, or fragments thereof, or antibodies, which are sufficient for treating a specific condition. Therapeutic efficacy and toxicity may be determined by standard pharmaceutical procedures in cell cultures is or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind a protein of the invention may be used for the diagnosis of conditions or diseases characterised by or associated with over- or underexpression of said protein of the invention, or in assays to monitor patients being treated with said protein of the invention, agonists, antagonists or inhibitors. The antibodies useful for diagnostic purposes may be prepared in the same manner as those described above for therapeutics. Diagnostic assays for a protein of the invention include methods, which utilise the antibody and a label to detect said protein of the invention in human body fluids or extracts of cells or tissues. The antibodies may be used with or without modification, and may be labelled by joining them, either covalently or non-covalently, with a reporter molecule. A wide variety of reporter molecules which are known in the art may be used several of which are described above.

A variety of protocols including ELISA, RIA, and FACS for measuring the proteins of the invention are known in the art and provide a basis for diagnosing altered or abnormal levels of expression of a protein of the invention. Normal or standard values for expression of the proteins of the invention are established by combining body fluids or cell extracts taken from normal mammalian subjects, preferably human, with antibody to a protein of the invention under conditions suitable for complex formation. The amount of standard complex formation may be quantified by various methods, but preferably by photometric means. Quantities of the proteins of the invention expressed in control and disease, samples e.g. from biopsied tissues are compared with the standard values. Deviation between standard and subject values establishes the parameters for diagnosing disease.

In another embodiment of the invention, the polynucleotides specific for a protein of the invention may be used for diagnostic purposes. The polynucleotides, which may be used, include oligonucleotide sequences, antisense RNA and DNA molecules, and PNAs. The polynucleotides may be used to detect and quantitate gene expression in biopsied tissues in which expression of said protein of the invention may be correlated with disease. The diagnostic assay may be used to distinguish between absence, presence, and excess expression of said protein of the invention, and to monitor regulation of the levels of said protein of the invention during therapeutic intervention.

In one aspect, hybridisation with PCR probes which are capable of detecting polynucleotide sequences, including genomic sequences, encoding a protein of the invention and homologous proteins or closely related molecules, may be used to identify nucleic acid sequences which encode respective protein. The hybridisation probes of the subject invention may be DNA or RNA and are preferably derived from the nucleotide sequence of the polynucleotide encoding the Drosophila or human proteins of the invention or from genomic sequence including promoter, enhancer elements, and introns of the naturally occurring genes. Hybridisation probes may be labelled by a variety of reporter groups, for example, radionuclides such as ³²p or ³⁵S, or enzymatic labels, such as alkaline phosphatase coupled to the probe via avidin/biotin coupling systems, and the like.

Polynucleotide sequences specific for the proteins of the invention and homologous proteins may be used for the diagnosis of conditions or diseases, which are associated with expression of a protein of the invention. Examples of such conditions or diseases include, but are not limited to, pancreatic diseases and disorders, including diabetes. Polynucleotide sequences encoding the proteins of the invention may also be used to monitor the progress of patients receiving treatment for pancreatic diseases and disorders, including diabetes. The polynucleotide sequences encoding the proteins of the invention may be used in Southern or Northern analysis, dot blot, or other membrane-based technologies; in PCR technologies; or in dip stick, pin, ELISA or chip assays utilising fluids or tissues from patient biopsies to detect altered gene expression.

In a particular aspect, the nucleotide sequences encoding the proteins of the invention may be useful in assays that detect activation or induction of various metabolic diseases such as obesity as well as related disorders such as eating disorder, cachexia, diabetes mellitus, hypertension, coronary heart disease, hypercholesterolemia, dyslipidemia, osteoarthritis, gallstones, cancers of the reproductive organs, and sleep apnea. The nucleotide sequences encoding the proteins of the invention may be labelled by standard methods, and added to a fluid or tissue sample from a patient under conditions suitable for the formation of hybridisation complexes. After a suitable incubation period, the sample is washed and the signal is quantitated and compared with a standard value. The presence of altered levels of nucleotide sequences encoding a protein of the invention in the sample indicates the presence of the associated disease. Such assays may also be used to evaluate the efficacy of a particular therapeutic treatment regimen in animal studies, in clinical trials, or in monitoring the treatment of an individual patient.

In order to provide a basis for the diagnosis of disease associated with expression of a protein of the invention, a normal or standard profile for expression is established. This may be accomplished by combining body fluids or cell extracts taken from normal subjects, either animal or human, with a sequence, or a fragment thereof, which encodes said protein of the invention, under conditions suitable for hybridisation or amplification. Standard hybridisation may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridisation assays may be repeated on a regular basis to evaluate whether the level of expression in the patient begins to approximate that, which is observed in the normal patient. The results obtained from successive assays may be used to show the efficacy of treatment over a period ranging from several days to months.

With respect to metabolic diseases such as obesity as well as related disorders such as eating disorder, cachexia, diabetes mellitus, hypertension, coronary heart disease, hypercholesterolemia, dyslipidemia, osteoarthritis, gallstones, cancers of the reproductive organs, and sleep apnea the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the pancreatic diseases and disorders. Additional diagnostic uses for oligonucleotides designed from the sequences encoding the proteins of the invention may involve the use of PCR. Such oligomers may be chemically synthesised, generated enzymatically, or produced from a recombinant source. Oligomers will preferably consist of two nucleotide sequences, one with sense orientation (5prime.fwdarw.3prime) and another with antisense (3prime.rarw.5prime), employed under optimised conditions for identification of a specific gene or condition. The same two oligomers, nested sets of oligomers, or even a degenerate pool of oligomers may be employed under less stringent conditions for detection and/or quantification of closely related DNA or RNA sequences.

In another embodiment of the invention, the nucleic acid sequences, which encode the proteins of the invention, may also be used to generate hybridisation probes, which are useful for mapping the naturally occurring genomic sequence. The sequences may be mapped to a particular chromosome or to a specific region of the chromosome using well known techniques. Such techniques include FISH, FACS, or artificial chromosome constructions, such as yeast artificial chromosomes, bacterial artificial chromosomes, bacterial P1 constructions or single chromosome cDNA libraries as reviewed in Price, C. M. (1993) Blood Rev. 7:127-134, and Trask, B. J. (1991) Trends Genet. 7:149-154. FISH (as described in Verma et al. (1988) Human Chromosomes: A Manual of Basic Techniques, Pergamon Press, New York, N.Y.) may be correlated with other physical chromosome mapping techniques and genetic map data. Examples of genetic map data can be found in the 1994 Genome Issue of Science (265:1981f). Correlation between the location of the gene encoding a protein of the invention on a physical chromosomal map and a specific disease, or predisposition to a specific disease, may help to delimit the region of DNA associated with that genetic disease.

The nucleotide sequences of the subject invention may be used to detect differences in gene sequences between normal, carrier, or affected individuals. An analysis of polymorphisms, e.g. single nucleotide polymorphisms, may be carried out. Further, in situ hybridisation of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localised by genetic linkage to a particular genomic region, for example, AT to 11q22-23 (Gatti, R. A. et al. (1988) Nature 336:577-580), any sequences mapping to that area may represent associated or regulatory genes for further investigation. The nucleotide sequences of the subject invention may also be used to detect differences in the chromosomal location due to translocation, inversion, etc. among normal, carrier, or affected individuals.

In another embodiment of the invention, the proteins of the invention, their catalytic or immunogenic fragments or oligopeptides thereof, can be used for screening libraries of compounds in any of a variety of drug screening techniques. One can identify effectors, e.g. receptors, enzymes, ligands, or substrates that bind to, modulate or mimic the action of one or more of the proteins of the invention. The protein or fragment thereof employed in such screening may be free in solution, affixed to a solid support, borne on a cell surface, or located intracellularly. The formation of binding complexes, between a protein of the invention and the agent tested, may be measured. Agents could also, either directly or indirectly, influence the activity of the proteins of the invention. For example the kinase or phosphatase activity of the proteins of the invention could be measured in vitro by using recombinantly expressed and purified Tec kinase, PTPRB or EDTP or fragments thereof by making use of artificial substrates well known in the art, i.e. but not exclusively DiFMUP (Molecular Probes, Eugene, Oreg.), which are converted to fluorophores or chromophores upon phosphorylation or dephosphorylation. Alternatively, the phosphorylation or dephosphorylation of physiological substrates of the kinases or phosphatases could be measured by making use of any of the well known screening technologies suitable for the detection of the phosphorylation status of their physiological substrates. For example, but not exclusively, the phosphorylation status of peptides derived from their physiological substrates can be monitored by binding of phospho-side specific antibodies resulting in an increase of the polarization of the complex.

In vivo, the enzymatic kinase activity of the unmodified polypeptides of Tec towards a substrate can be enhanced by appropriate stimuli, triggering the phosphorylation of Tec. This may be induced in the natural context by extracellular or intracellular stimuli, such as signaling molecules or environmental influences. One may generate a system containing actived Tec, may it be an organism, a tissue, a culture of cells or cell-free environment, by exogenously applying this stimulus or by mimicking this stimulus by a variety of the techniques, some of them described further below. A system containing activated Tec may be produced (i) for the purpose of diagnosis, study, prevention, and treatment of diseases and disorders related to body-weight regulation and thermogenesis, for example, but not limited to, metabolic diseases such as obesity, as well as related disorders such as eating disorder, cachexia, diabetes mellitus, hypertension, coronary heart disease, hypercholesterolemia, dyslipidemia, osteoarthritis, gallstones, and sleep apnea, (ii) for the purpose of identifying or validating therapeutic candidate agents, pharmaceuticals or drugs that influence the genes of the invention or their encoded polypeptides, (iii) for the purpose of generating cell lysates containing activated polypeptides encoded by the genes of the invention, (iv) for the purpose of isolating from this source activated polypeptides encoded by the genes of the invention.

In addition activity of PTPRB, EDTP, or tec against its physiological substrate(s) or derivatives thereof can be measured in cell-based assays. Agents may also interfere with posttranslational modifications of the protein, such as phosphorylation and dephosphorylation, acetylation, alkylation, ubiquitination, proteolytic processing, subcellular localization, or degradation. Moreover, agents can influence the dimerization or oligomerization of the proteins of the invention or, in a heterologous manner, of the proteins of the invention with other proteins, for example, but not exclusively, docking proteins, enzymes, receptors, or translation factors. Agents can also act on the physical interaction of the proteins of this invention with other proteins, which are required for protein function, for example, but not exclusively, their downstream signalling. Methods for determining protein-protein interaction are well known in the art. For example binding of a fluorescently labeled peptide derived from the interacting protein to the protein of the invention, or vice versa, could be detected by a change in polarisation. In case that both binding partners, which can be either the full length proteins as well as one binding partner as the full length protein and the other just represented as a peptide are fluorescently labeled, binding can be detected by fluorescence energy transfer (FRET) from one fluorophore to the other. In addition, a variety of commercially available assay principles suitable for detection of protein-protein interaction are well known In the art, for example but not exclusively AlphaScreen (PerkinElmer) or Scintillation Proximity Assays (SPA) by Amersham. Alternatively, the interaction of the proteins of the invention with cellular proteins can be the basis for a cell-based screening assay, in which both proteins are fluorescently labeled and interaction of both proteins is detected by analysing cotranslocation of both proteins with a cellular imaging reader, as has been developed for example, but not exclusively, by Cellomics or EvotecOAI. In all cases the two or more binding partners can be different proteins with one being the protein of the invention, or in case of dimerization and/or oligomerization the protein of the invention itself. Proteins of the invention, for which one target mechanism of interest, but not the only one, would be such protein/protein interactions are PTPRB, Tec, or EDTP.

Of particular interest are screening assays for agents that have a low toxicity for mammalian cells. The term “agent” as used herein describes any molecule, e.g. protein or pharmaceutical, with the capability of altering or mimicking the physiological function of one or more of the proteins of the invention. Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Candidate agents are also found among biomolecules including peptides, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal. Another technique for drug screening, which may be used, provides for high throughput screening of compounds having suitable binding affinity to the protein of interest as described in published PCT application WO84/03564. In this method, as applied to the proteins of the invention large numbers of different small test compounds, e.g. aptamers, peptides, low-molecular weight compounds etc., are provided or synthesized on a solid substrate, such as plastic pins or some other surface. The test compounds are reacted with the proteins or fragments thereof, and washed. Bound proteins are then detected by methods well known in the art. Purified proteins can also be coated directly onto plates for use in the aforementioned drug screening techniques. Alternatively, non-neutralizing antibodies can be used to capture the peptide and immobilize it on a solid support. In another embodiment, one may use competitive drug screening assays in which neutralizing antibodies capable of binding the protein specifically compete with a test compound for binding the protein. In this manner, the antibodies can be used to detect the presence of any peptide, which shares one or more antigenic determinants with the protein.

The nucleic acids encoding the proteins of the invention can be used to generate transgenic cell lines and animals. These transgenic non-human animals are useful in the study of the function and regulation of the proteins of the invention in vivo. Transgenic animals, particularly mammalian transgenic animals, can serve as a model system for the investigation of many developmental and cellular processes common to humans. A variety of non-human models of metabolic disorders can be used to test modulators of the protein of the invention. Misexpression (for example, overexpression or lack of expression) of the protein of the invention, particular feeding conditions, and/or administration of biologically active compounts can create models of metablic disorders.

In one embodiment of the invention, such assays use mouse models of insulin resistance and/or diabetes, such as mice carrying gene knockouts in the leptin pathway (for example, ob (leptin) or db (leptin receptor) mice). Such mice develop typical symptoms of diabetes, show hepatic lipid accumulation and frequently have increased plasma lipid levels (see Bruning et al, 1998, Mol. Cell. 2:449-569). Susceptible wild type mice (for example C57BI/6) show similiar symptoms if fed a high fat diet. In addition to testing the expression of the proteins of the invention in such mouse strains (see EXAMPLE 4), these mice could be used to test whether administration of a candidate modulator alters for example lipid accumulation in the liver, in plasma, or adipose tissues using standard assays well known in the art, such as FPLC, calorimetric assays, blood glucose level tests, insulin tolerance tests and others.

Transgenic animals may be made through homologous recombination in embryonic stem cells, where the normal locus of the gene encoding the protein of the invention is mutated. Alternatively, a nucleic acid construct encoding the protein is injected into oocytes and is randomly integrated into the genome. One may also express the genes of the invention or variants thereof in tissues where they are not normally expressed or at abnormal times of development. Furthermore, variants of the genes of the invention like specific constructs expressing anti-sense molecules or expression of dominant negative mutations, which will block or alter the expression of the proteins of the invention may be randomly integrated into the genome. A detectable marker, such as lac Z or luciferase may be introduced into the locus of the genes of the invention, where upregulation of expression of the genes of the invention will result in an easily detectable change in phenotype. Vectors for stable integration include plasmids, retroviruses and other animal viruses, yeast artificial chromosomes (YACs), and the like. DNA constructs for homologous recombination will contain at least portions of the genes of the invention with the desired genetic modification, and will include regions of homology to the target locus. Conveniently, markers for positive and negative selection are included. DNA constructs for random integration do not need to contain regions of homology to mediate recombination. DNA constructs for random integration will consist of the nucleic acids encoding the proteins of the invention, a regulatory element (promoter), an intron and a poly-adenylation signal. Methods for generating cells having targeted gene modifications through homologous recombination are known in the field. For embryonic stem (ES) cells, an ES cell line may be employed, or embryonic cells may be obtained freshly from a host, e.g. mouse, rat, guinea pig, etc. Such cells are grown on an appropriate fibroblast-feeder layer and are grown in the presence of leukemia inhibiting factor (LIF). ES or embryonic cells may be transfected and can then be used to produce transgenic animals. After transfection, the ES cells are plated onto a feeder layer in an appropriate medium. Cells containing the construct may be selected by employing a selection medium. After sufficient time for colonies to grow, they are picked and analyzed for the occurrence of homologous recombination. Colonies that are positive may then be used for embryo manipulation and morula aggregation. Briefly, morulae are obtained from 4 to 6 week old superovulated females, the Zona Pellucida is removed and the morulae are put into small depressions of a tissue culture dish. The ES cells are trypsinized, and the modified cells are placed into the depression closely to the morulae. On the following day the aggregates are transfered into the uterine horns of pseudopregnant females. Females are then allowed to go to term. Chimeric offsprings can be readily detected by a change in coat color and are subsequently screened for the transmission of the mutation into the next generation (F1-generation). Offspring of the F1-generation are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc., for example, mouse, rat, guinea pig, sheep, cow, pig, and others. The transgenic animals may be used in functional studies, drug screening, and other applications and are useful in the study of the function and regulation of the proteins of the invention in vivo.

Finally, the invention also relates to a kit comprising at least one of
(a) a nucleic acid molecule encoding one of the proteins of the invention or a fragment or an isoform thereof;
(b) an amino acid molecule of a protein described in this invention or a fragment or an isoform thereof:
(c) a vector comprising the nucleic acid of (a);
(d) a host cell comprising the nucleic acid of (a) or the vector of (b);
(e) a polypeptide encoded by the nucleic acid of (a), expressed by the vector of (c) or the host cell of (d);
(f) a fusion polypeptide encoded by the nucleic acid of (a);
(g) an antibody, an aptamer or another receptor against the nucleic acid of (a) or the polypeptide of (b), (e) or (f) and
(h) an anti-sense oligonucleotide of the nucleic acid of (a).

The kit may be used for diagnostic or therapeutic purposes or for screening applications as described above. The kit may further contain user instructions.

The Figures show:

FIG. 1 shows the triglyceride content of Drosophila Btk29A (GadFly Accession Number CG18355, formerly CG8049) mutants. Shown is the change of triglyceride of EP(2)0715 flies caused by homozygous viable integration of the P-vector into the annotated transcription unit (column 3) in comparison to controls containing all fly lines of the EP collection (‘EP-control males’, column 1).

FIG. 2 shows the molecular organisation of the mutated Btk29A gene locus.

FIG. 3 shows the human sequences homologous to Btk29A.

FIG. 3A shows the nucleic acid sequence of human tec protein tyrosine kinase (SEQ ID NO: 1).

FIG. 3B shows the amino acid sequence (one-letter code) of human tec protein tyrosine kinase (SEQ ID NO: 2).

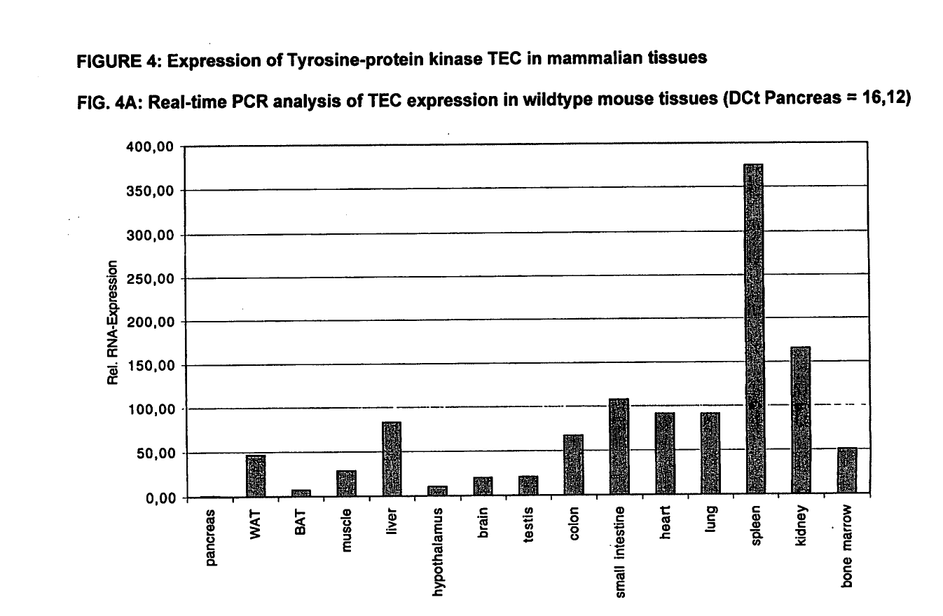

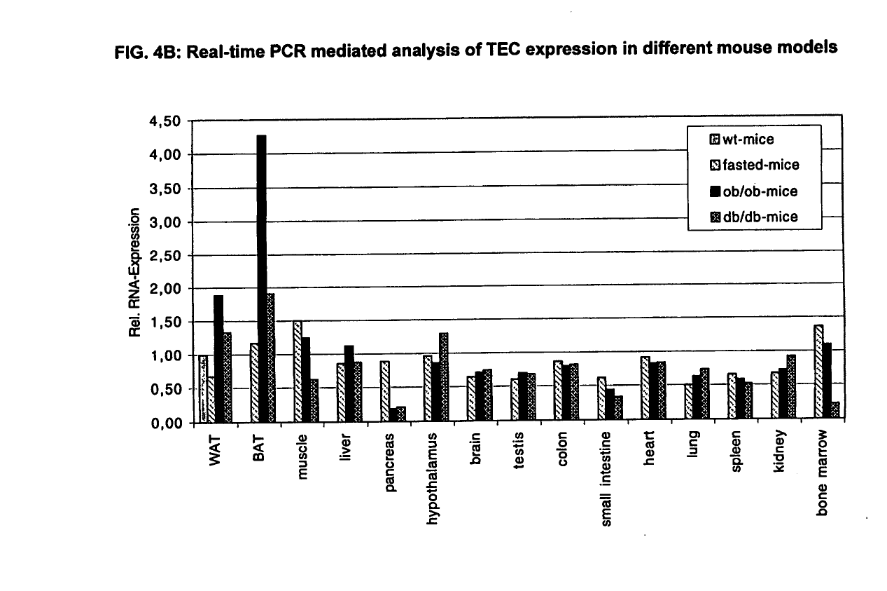

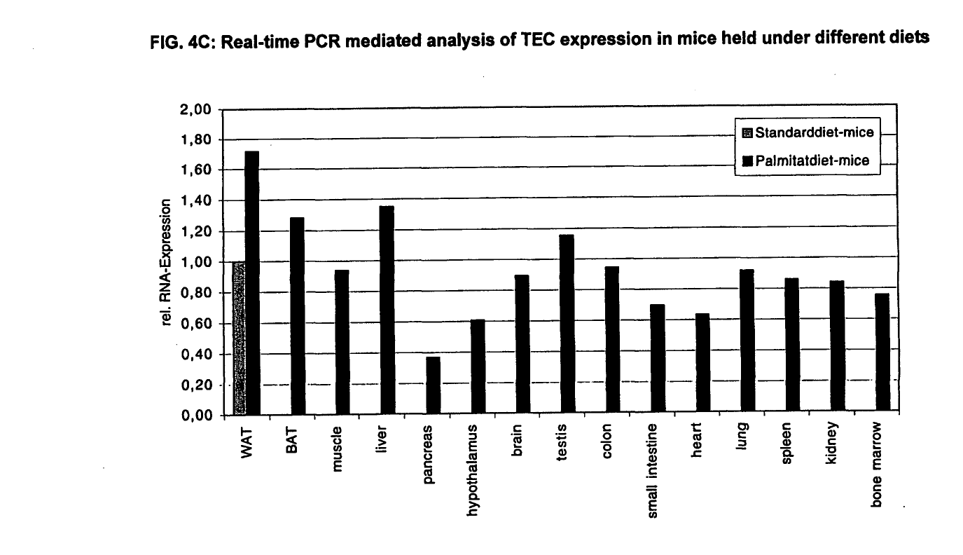

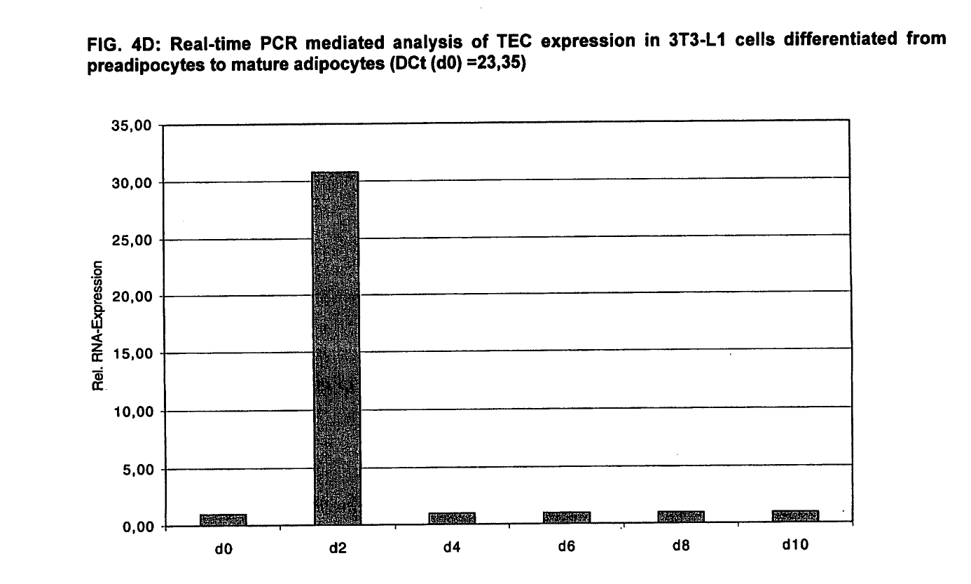

FIG. 4 shows the expression of tec tyrosine protein kinase in different mammalian models

FIG. 4A. Real-time PCR analysis of tec tyrosine protein kinase expression in wildtype mouse tissues. The relative RNA-expression is shown on the Y-axis, the tissues tested are given on the X-axis. WAT=white adipose tissue, BAT=brown adipose tissue.

FIG. 4B shows real-time PCR analysis of tec tyrosine protein kinase expression in different tissues of wildtype mice (wt-mice), of fasted mice (fasted-mice), of genetically obese mice (ob/ob-mice and db/db-mice). The relative RNA-expression is shown on the Y-axis, the tissues tested are given on the X-axis. WAT=white adipose tissue, BAT=brown adipose tissue.

FIG. 4C shows the real-time PCR analysis of tec tyrosine protein kinase expression in different tissues of wildtype mice, held under different diets (Standarddiet-mice and Palmitatdiet-mice). The relative RNA-expression is shown on the Y-axis, the tissues tested are given on the X-axis. WAT=white adipose tissue, BAT=brown adipose tissue.

FIG. 4D shows the real-time PCR mediated comparison of tec tyrosine protein kinase expression during differentiation of mammalian fibroblast (3T3-L1) cells from pre-adipocytes to mature adipocytes.

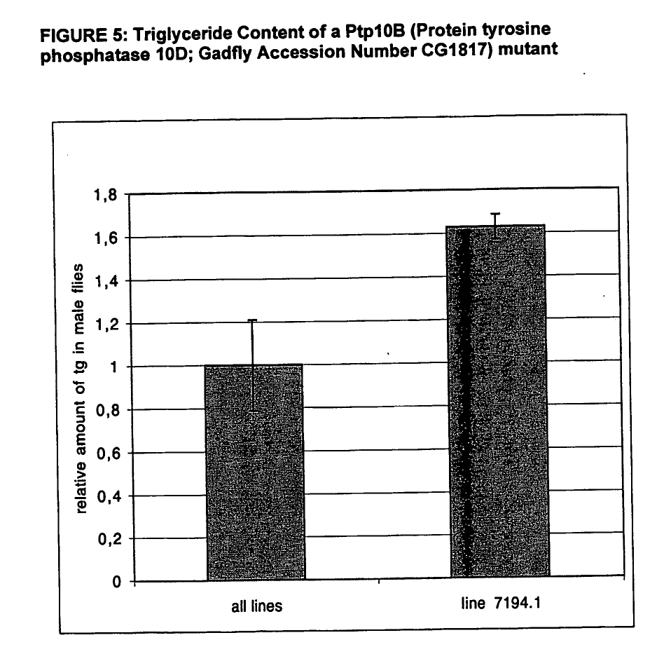

FIG. 5 shows the relative triglyceride content (triglyceride/weight) of Drosophila PTP10B (GadFly Accession Number CG1817) mutants. Shown is the change of triglyceride of PX7194.1 flies caused by hemizygous viable integration of the P-vector into the annotated transcription unit (column 2) in comparison to controls containing all fly lines of the PX mutant collection (‘all lines’, column 1).

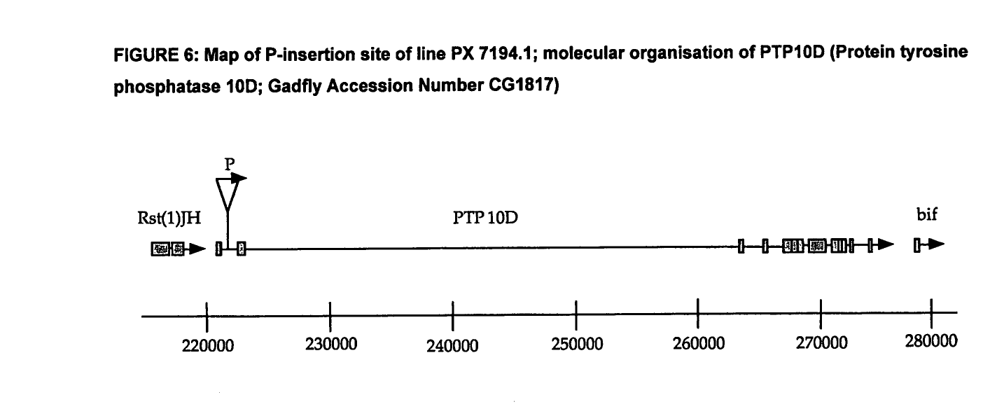

FIG. 6 shows the molecular organisation of the mutated PTP10D gene locus. In the map of the P-insertion site of line PX7194.1, PTP10D referres to Receptor Protein Tyrosine Phosphatase 10D encoding gene, bif referres to bifocal, and Rst(1)JH referrs to Resistance to Juvenile Hormone.

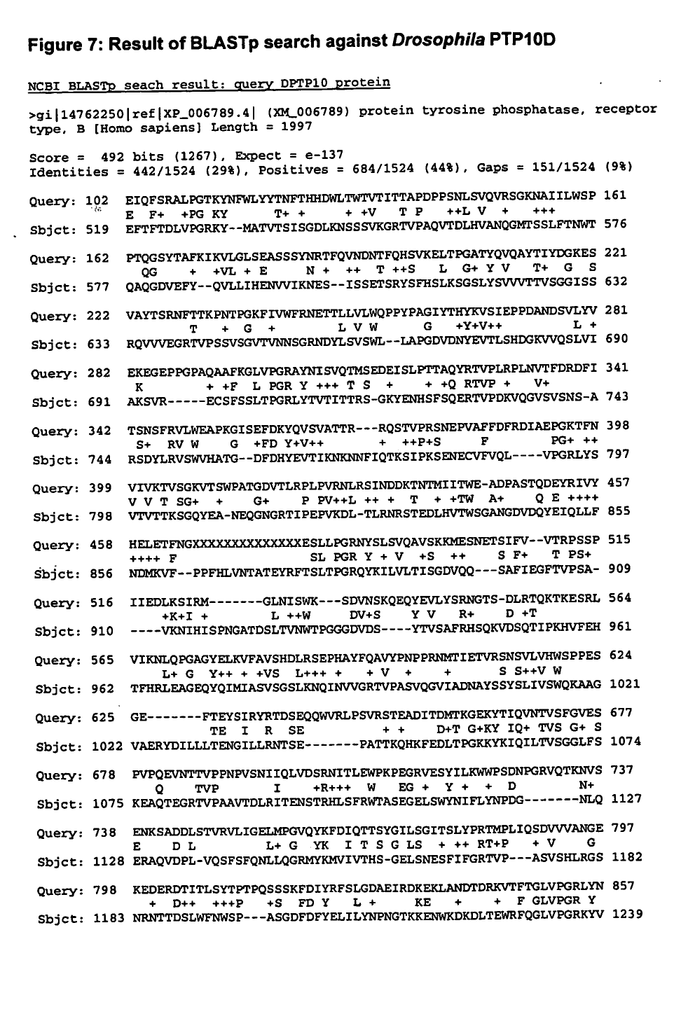

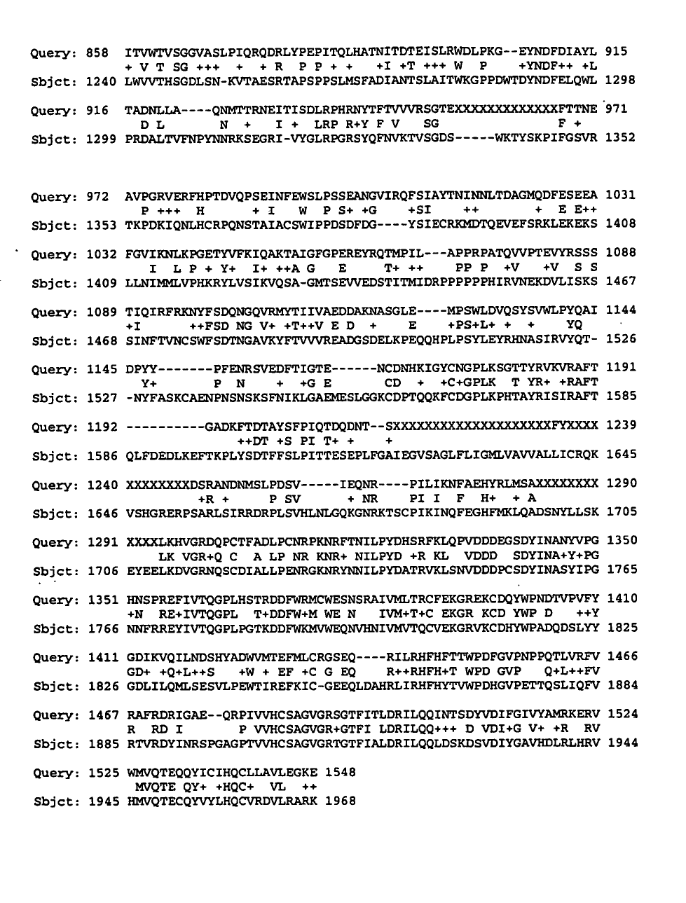

FIG. 7 shows the results of a BLASTp search against Drosophila PTP10D.

FIG. 8 shows the human PTP10D homologous sequences

FIG. 8A shows the nucleic acid sequence of human protein tyrosine phosphatase, receptor type, B (PTPRB) (SEQ ID NO: 3).

FIG. 8B shows the amino acid sequence (one-letter code) of human protein tyrosine phosphatase, receptor type, B (PTPRB) (SEQ ID NO: 4).

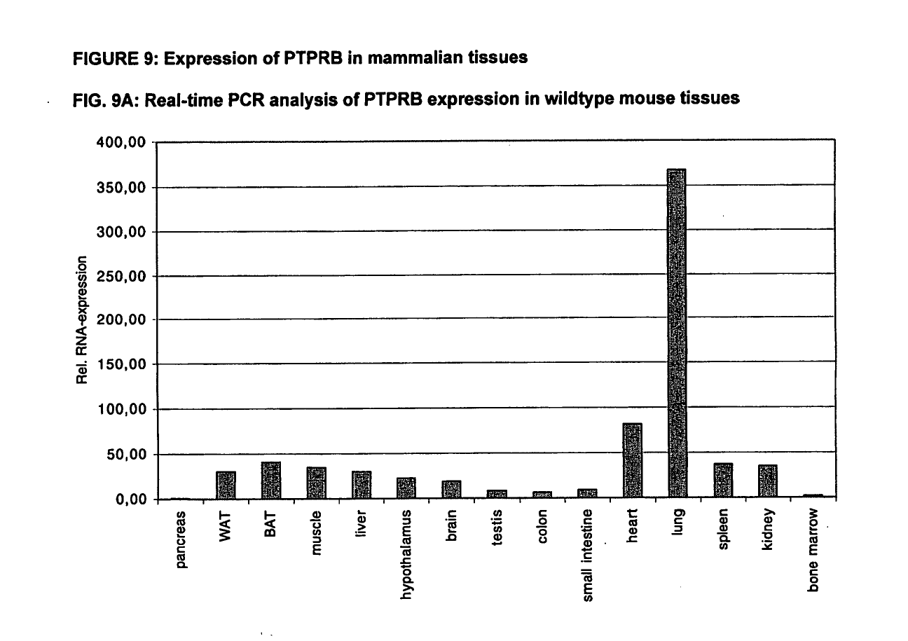

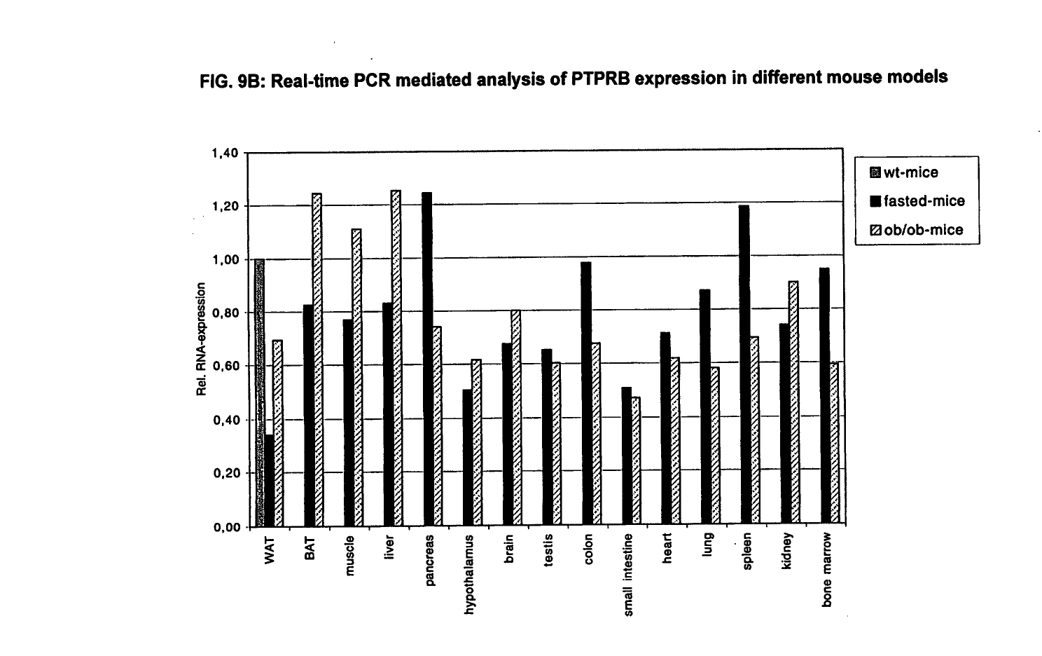

FIG. 9 shows the expression of the PTPRB gene in mammalian tissues.

FIG. 9A shows the real-time PCR analysis of PTPRB expression in wildtype mouse tissues. The relative RNA-expression is shown on the Y-axis, the tissues tested are given on the X-axis. WAT=white adipose tissue, BAT=brown adipose tissue.

FIG. 9B shows the real-time PCR analysis of PTPRB expression in different tissues of wildtype mice (wt-mice), of fasted mice (fasted-mice), and of genetically obese mice (ob/ob-mice). The relative RNA-expression is shown on the Y-axis, the tissues tested are given on the X-axis. WAT=white adipose tissue, BAT=brown adipose tissue.

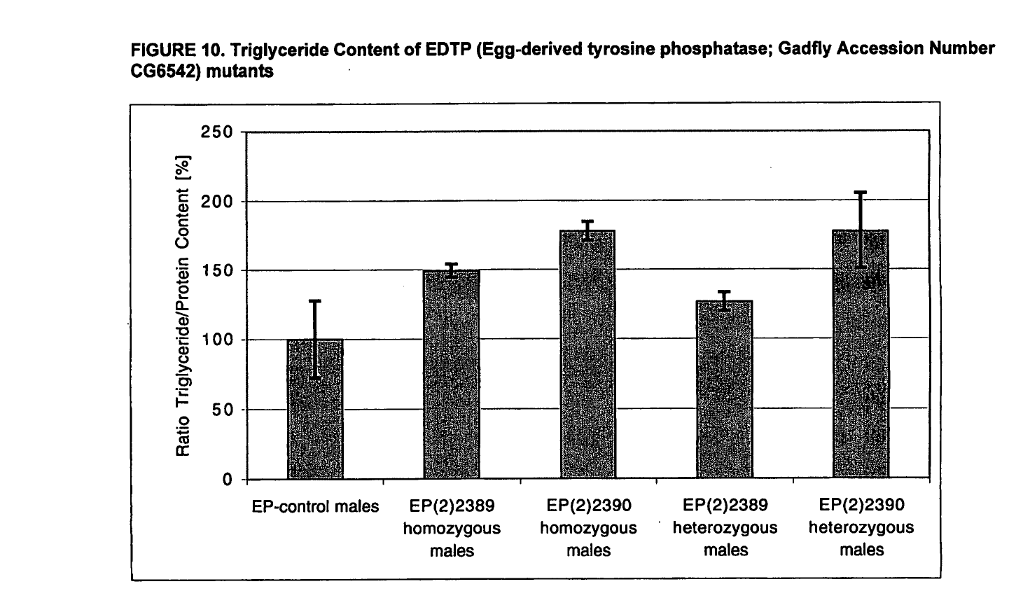

FIG. 10 shows the triglyceride content of Drosophila EDTP (GadFly Accession Number CG6542) mutants. Shown is the change of triglyceride of EP(2)2389 flies and EP(2)2390 flies caused by homozygous viable and heterozygous integration of the P-vectors into the annotated transcription unit (columns 2, 3, 4, and 5) in comparison to controls containing all fly lines of the EP collection (‘EP-control males’, column 1).

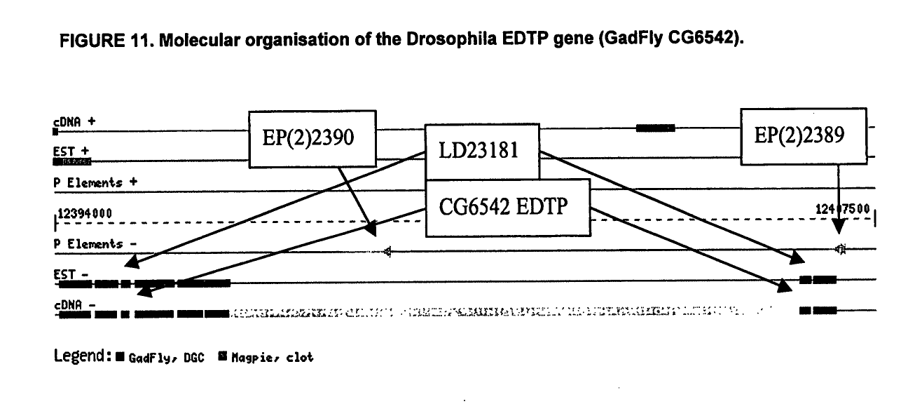

FIG. 11 shows the molecular organisation of the mutated EDTP gene locus.

FIG. 12 shows the human EDTP homologous sequences

FIG. 12A shows the nucleic acid sequence encoding the human homolog of EDTP (SEQ ID NO: 5).

FIG. 12B shows the amino acid sequence (one-letter code) of the human homolog of EDTP (GenBank Accession Number CAD28494.1) (SEQ ID NO: 6).

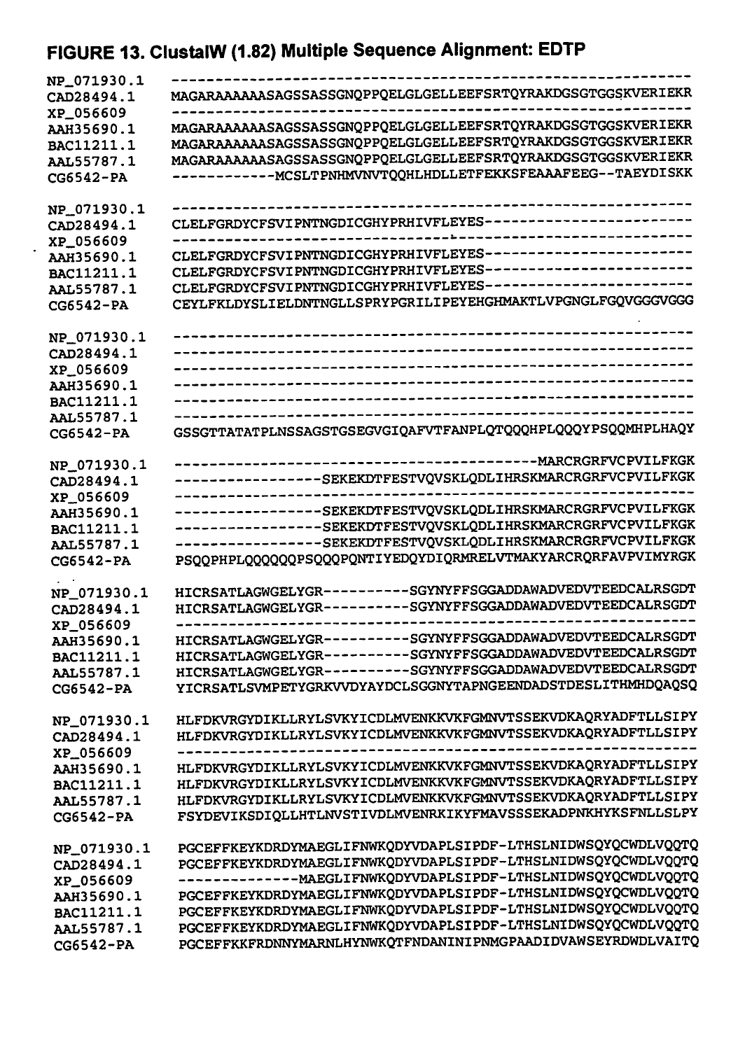

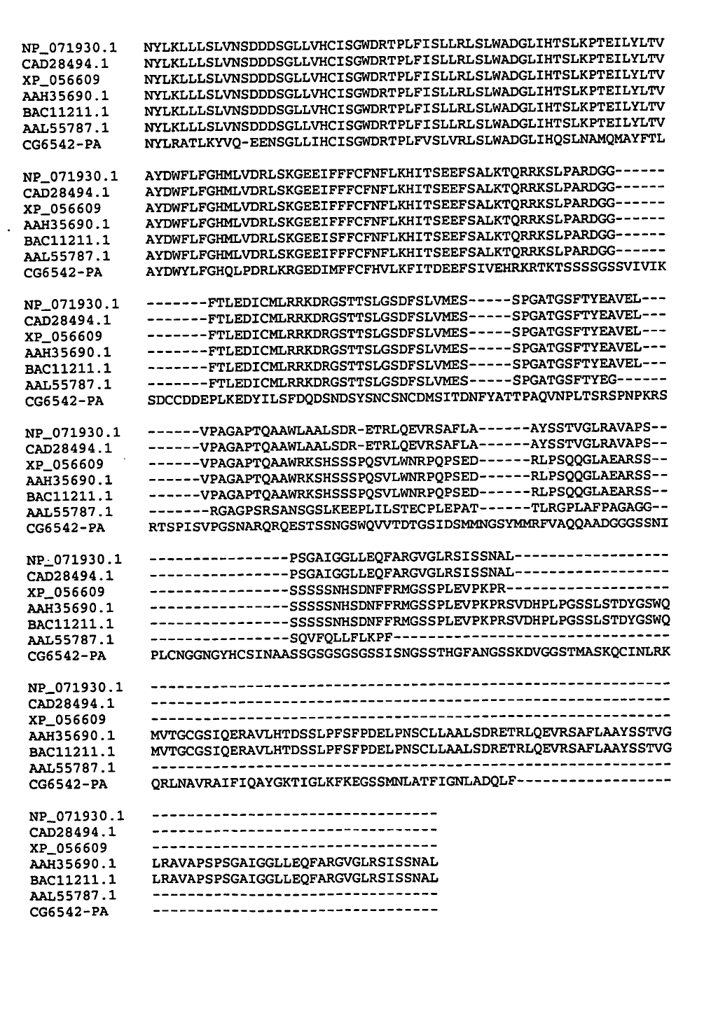

FIG. 13 shows the comparison (CLUSTAL W 1.82 multiple sequence alignment) of EDTP proteins from different species, CG6542-PA refers to a protein isoform encoded by Drosophila EDTP gene with GadFly Accession No. CG6542, NP_{—}071930.1, CAD28494.1, XP_{—}056609, AAH3569.1, BAC11211.1, and AAL55787.1 refer to the GenBank Accession Numbers of human homologs of EDTP. Gaps in the alignment are represented as −.

The examples illustrate the invention:

### EXAMPLE 1

### Measurement of Triglyceride Content in Drosophila

The change of triglyceride content of Drosophila melanogaster containing a special expression system (EP-element; Rorth P., (1996) Proc Natl Acad Sci USA 93(22):12418-12422) was measured. Mutant flies are obtained from fly mutation stock collections (proprietary fly mutation stock collection; P insertion Mutation Stock Center, Sezged, Hungary). The flies are grown under standard conditions known to those skilled in the art. In the course of the experiment, additional feedings with bakers yeast (Saccharomyces cerevisiae) are provided. The average increase of triglyceride content of Drosophila containing the EP(2)0715, PX7194.1, EP(2)2389, or EP(2)2390 vectors in homozygous viable, hemizygous viable, and heterozygous integration was investigated in comparison to control flies (FIGS. 1, 5, and 10). For determination of triglyceride, flies were incubated for 5 min at 90° C. in an aqueous buffer using a waterbath, followed by hot extraction. After another 5 min incubation at 90° C. and mild centrifugation, the triglyceride content of the flies extract was determined using Sigma Triglyceride (INT 336-10 or -20) assay by measuring changes in the optical density according to the manufacturer's protocol. As a reference protein content of the same extract was measured using BIO-RAD DC Protein Assay according to the manufacturer's protocol, or the weight of the flies was measured. The assays were repeated three times. The average triglyceride level of all flies of the EP collection (referred to as ‘EP-control males’ in FIGS. 1 and 10) or the PX collection (referred to as ‘all lines’ in FIG. 5) is shown as 100% in FIGS. 1, 5, and 10.

EP(2)0715 homozygous flies show constantly a higher triglyceride content than the controls (approx. 60%; column 2 in FIG. 1). Therefore, the loss of gene activity in the locus 29A1-3 on chromosome 2L where the EP-vector of EP(2)0715 flies is homozygous viable integrated, is responsible for changes in the metabolism of the energy storage triglycerides, therefore representing in both cases an model for obese flies.

PX7194.1 hemizygous flies show constantly a higher triglyceride content than the controls (approx. 60%; column 2 in FIG. 5). Therefore, the change of gene activity in the locus of the PX7194.1 integration on chromosome X where the PX-vector of PX7194.1 flies is hemizygous viable integrated, is responsible for changes in the metabolism of the energy storage triglycerides.

EP(2)2389 homozygous flies show constantly a higher triglyceride content than the controls (approx. 50%; column 2 in FIG. 10), EP(2)2389 heterozygous flies also show constantly a higher triglyceride content than the controls (approx. 25%, as shown in column 4 in FIG. 10), and EP(2)2390 homozygous and heterozygous flies show constantly a higher triglyceride content than the controls (approx. 75%, as shown in columns 3 and 5 in FIG. 10, respectively). Therefore, the loss of gene activity in the locus 54C1-54C3 where the EP-vector of EP(2)2389 or EP(2)2390 flies is homozygous or heterozygous viably integrated, is responsible for changes in the metabolism of the energy storage triglycerides.

The increase of triglyceride content due to the potential loss of a gene function suggests potential gene activities in energy homeostasis in a dose dependent manner that controls the amount of energy stored as triglycerides.

### EXAMPLE 2

### Identification of Drosophila Genes Responsible for Changes in Triglyceride Levels

Using the iPCR method, genomic DNA sequences were isolated that are localised directly adjacent in 3′ direction of the integration site of the EP vectors (herein EP(2)0715, EP(2)2389, or EP(2)2390) and that are localised directly adjacent in 5′ direction of the integration site of the EP vectors (herein PX7194.1). Using those isolated genomic sequences public databases like Berkeley Drosophila Genome Project (GadFly) were screened thereby identifying the integration sites of the vectors. FIGS. 2, 6, and 11 show the molecular organisation of these gene loci.

The chromosomal localization site of the integration of the vector in line EP(2)0715 is at gene locus 2L, 29A1-3. In FIG. 2, genomic DNA sequence is represented by the assembly as a dotted black line (from position 8157113 to 8182113 on chromosome 2L) that includes the integration sites of vector for line EP(2)0715. Transcribed DNA sequences (ESTs) and predicted exons are shown as bars in the lower two lines. Predicted exons of the cDNA with GadFly Accession Number CG8049 are shown as dark grey bars and introns as light grey bars. Tec encodes for a gene that is predicted by GadFly sequence analysis programs as Accession Number CG8049. Public DNA sequence databases (for example, NCBI GenBank) were screened thereby identifying the integration sites of lines EP(2)0715, causing an increase of triglyceride content. EP(2)0715 is integrated into the promoter region 5′ in antisense orientation of the cDNA with Gadfly Accession Number CG8049. Therefore, expression of the cDNA encoding Btk29A (Gadfly Accession Number CG8049) could be effected by homozygous integration of vectors of line EP(2)0715, leading to an increase of the energy storage triglycerides.

The chromosomal localization site of the integration of the vector in line PX7194.1 is at gene locus X, 10C7-D1. In FIG. 6, numbers represent the coordinates of the genomic DNA (starting at position 220000 on chromosome X, ending at position 280000 on chromosome X). Grey bars predicted exons of the genes (as predicted by the Berkeley Drosophila Genome Project, GadFly and by Magpie). The integration site of the vector for line PX7194.1 is indicated at position 222666. PTP10D encodes for a gene that has been identified and cloned by Tian, S et al. (1991) (1991) Cell 67:675 and Yang, X. et al. (1991) Cell 67:661. Using the published sequences of the PTP10D encoding gene, the hemizygous viable integration site of the PX7194.1 vector has been determined. It is located in an intron between the first and second exon of the PTP10D gene (5′ to the start codon), causing an increase of triglyceride content.

The chromosomal localization site of the integration of the vectors in lines EP(2)2389 and EP(2)2390 is at gene locus 2R, 54C1-54-C3. In FIG. 11, genomic DNA sequence is represented by the assembly as a dotted black line (from position 1239400 to 12407500 on chromosome 2R) that includes the integration sites of vector for lines EP(2)2389 and EP(2)2390. Transcribed DNA sequences (ESTs) and predicted exons are shown as bars in the lower two lines. Predicted exons of the cDNA with GadFly Accession Number CG6542 are shown as dark grey bars and introns as light grey bars. EDTP encodes for a gene that is predicted by GadFly sequence analysis programs as Accession Number CG6542. Public DNA sequence databases (for example, NCBI GenBank) were screened thereby identifying the integration sites of lines EP(2)2389 or EP(2)2390, causing an increase of triglyceride content. EP(2)2389 is viably integrated 100 base pairs of in the 5′ exon of the cDNA with Accession Number CG6542, EP(2)2390 is integrated in the second exon of the cDNA with Accession Number CG6542. Therefore, expression of the cDNA encoding Accession Number CG6542 could be effected by homozygous or heterozygous viable integration of vectors of lines EP(2)2389 or EP(2)2390, leading to increase of the energy storage triglycerides.

### EXAMPLE 3

### Identification of Human PTP10D, Tec, and EDTP Homologues

The proteins of the invention and homologous proteins and nucleic acid molecules coding therefore are obtainable from insect or vertebrate species, e.g. mammals or birds. Particularly preferred are nucleic acids encoding the Drosophila and human homologs of the proteins of the invention. Sequences homologous to Drosophila proteins of the invention were identified using the publicly available program BLASTP 2.2.3 of the non-redundant protein data base of the National Center for Biotechnology Information (NCBI) (see, Altschul et. Al., (1997), Nucleic Acids Res. 25:3389-2402).

Particularly preferred are nucleic acids encoding Drosophila Btk29A (GadFly Accession Numbers CG18355, CG8049), human tec protein tyrosine kinase (Genbank Accession Number NM-003215.1 (formerly XM_{—}044444.3) for the cDNA (SEQ ID NO: 1, see FIG. 3A), Genbank Accession Number NP_{—}003206.1 (formerly XP_{—}044444) for the protein (SEQ ID NO: 2, see FIG. 3B), and mouse protein tyrosine kinase TecIV (Genbank Accession Number AAD43402.1). An alignment of Tec from different species has been done by the ClustaW program.

Particularly preferred are nucleic acids encoding Drosophila PTP10D (Gadfly Accession Number CG1817), human PTPRB (Genbank Accession Number XM 006789.7 for the cDNA (SEQ ID NO: 3, see FIG. 8A), Genbank Accession Number XP_{—}006789.4 for the protein (SEQ ID NO: 4, see FIG. 8B), and mouse receptor-type protein tyrosine phosphatase (Genbank Accession Number AF157628 for the cDNA, Genbank Accession Number AAF80346 for the protein).

Particularly preferred are nucleic acids encoding Drosophila EDTP (Gadfly Accession Number CG6542), human EDTP homologous proteins on chromosome 3 (Genbank Accession Numbers XM_{—}056609.3 and NM_{—}022485.2 for the cDNAs, XP_{—}056609 and NP_{—}071930 for the proteins), cDNA of SEQ ID NO: 5 (FIG. 12A) encoding protein of SEQ ID NO: 6 (FIG. 12B), predicted cDNA from Genbank Accession Numbers, XM_{—}056748, and BC_{—}001674, further human proteins with Genbank Accession Numbers AAH35690.1, AAL55787, BAC11211, and CAD28494).

### EXAMPLE 4

### Expression of the Polypeptides in Mammalian (Mouse) Tissues

For analyzing the expression of the polypeptides disclosed in this invention in mammalian tissues, several mouse strains (preferably mice strains C57BI/6J, C57BI/6 ob/ob and C57BI/KS db/db which are standard model systems in obesity and diabetes research) were purchased from Harlan Winkelmann (33178 Borchen, Germany) and maintained under constant temperature (preferably 22° C.), 40 percent humidity and a light/dark cycle of preferably 14/10 hours. The mice were fed a standard chow (for example, from ssniff Spezialitaten GmbH, order number ssniff M-Z V 126-000). For the fasting experiment (“fasted wild type mice”), wild type mice were starved for 48 h without food, but only water supplied ad libitum. (see, for example, Schnetzler et al. J Clin Invest 1993 July;92(1):272-80, Mizuno et al. Proc Natl Acad Sci USA 1996 Apr. 16;93(8):3434-8). Animals were sacrificed at an age of 6 to 8 weeks. The animal tissues were isolated according to standard procedures known to those skilled in the art, snap frozen in liquid nitrogen and stored at −80° C. until needed.

For analyzing the role of the proteins disclosed in this invention in the in vitro differentiation of different mammalian cell culture cells for the conversion of pre-adipocytes to adipocytes, mammalian fibroblast (3T3-L1) cells (e.g., Green & Kehinde, Cell 1: 113-116, 1974) were obtained from the American Tissue Culture Collection (ATCC, Hanassas, Va., USA; ATCC-CL 173). 3T3-L1 cells were maintained as fibroblasts and differentiated into adipocytes as described in the prior art (e.g., Qiu, et al., J. Biol. Chem. 276:11988-95, 2001; Slieker et al., BBRC 251: 225-9, 1998). In brief, cells were plated in DMEM/10% FCS (Invitrogen, Karlsruhe, Germany) at 50,000 cells/well in duplicates in 6-well plastic dishes and cultured in a humidified atmosphere of 5% CO₂ at 37° C. At confluence (defined as day 0: dO) cells were transferred to serum-free (SF) medium, containing DMEM/HamF12 (3:1; Invitrogen), Fetuin (300 μg/ml; Sigma, Munich, Germany), transferrin (2 μg/ml; Sigma), pantothenate (17 μM; Sigma), biotin (1 μM; Sigma), and EGF (0.8 nM; Hoffmann-La Roche, Basel, Switzerland). Differentiation was induced by adding dexamethasone (DEX; 1 μM; Sigma), 3-methyl-isobutyl-1-methylxanthine (MIX; 0.5 mM; Sigma), and bovine insulin (5 μg/ml; Invitrogen). Four days after confluence (d4), cells were kept in SF medium, containing bovine insulin (5 μg/ml) until differentiation was completed. At various time points of the differentiation procedure, beginning with day 0 (day of confluence) and day 2 (hormone addition; for example, dexamethasone and 3-isobutyl-1-methylxanthine), up to 10 days of differentiation, suitable aliquots of cells were taken every two days.

At various time points of the differentiation procedure, beginning with day 0 (day of confluence and hormone addition, for example, insulin), up to 10 days of differentiation, suitable aliquots of cells were taken every two days.

Expression Profile Analysis of the Proteins of the Invention

RNA was isolated from mouse tissues or cell culture cells using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art. Total RNA was reverse transcribed (preferably using Superscript II RNaseH-Reverse Transcriptase, from Invitrogen, Karlsruhe, Germany) and subjected to Taqman analysis preferably using the Taqman 2×PCR Master Mix (from Applied Biosystems, Weiterstadt, Germany; the Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (from Applied Biosystems, Weiterstadt, Germany).

For the analysis of the expression of Tec, taqman analysis was performed using the following primer/probe pairs:
Mouse Tec forward primer (Seq ID NO: 7) 5′-TCT TGG CTT GTC TCG GCA C-3′;
Mouse Tec reverse primer (Seq ID NO: 8) 5′-GGA CAT CCT CTG TGT CCT CAG TG-3′;
Mouse Tec Taqman probe (Seq ID NO: 9) (5/6-FAM) CGC TCC GTC AAG GTG TCG GAC G (5/6-TAMRA)

For the analysis of the expression of PTPRB, taqman analysis was performed using the following primer/probe pairs:
Mouse PTPRB forward primer (Seq ID NO: 10) 5′-GAG AAT ACA TCG CCA CTC AGG G-3′;
Mouse PTPRB reverse primer (Seq ID NO: 11) 5′-CTC CCA CGC CAT CTT CCA-3′;
Mouse PTPRB Taqman probe (Seq ID NO: 12) (5/6-FAM) CGC TTC CAG GCA CCA AGG ATG ACT T (5/6-TAMRA)

Expression profiling studies confirm the particular relevance of Tec kinase as regulator of enery metabolism in mammals. Tec transcripts are expressed in several wildtype tissues of mammals, and is clearly expressed in white adipose tissue (WAT) (see FIG. 4A), indicating a role in the regulation of energy homeostasis.

Further, we show that the mammalian homologue of the Drosophilia Tec gene is regulated in genetically induced obesity. In this invention, we used mouse models of insulin resistance and/or diabetes, such as mice carrying gene knockouts in the leptin pathway (for example, ob (leptin) mice) to study the expression of the protein of the invention. Such mice develop typical symptoms of diabetes, show hepatic lipid accumulation and frequently have increased plasma lipid levels (see Bruning et al, 1998, Mol. Cell. 2:449-569). We observed a marked upregulation in the metabolically active tissue (for example, brown adipose tissue (BAT)) as well as white adipose tissue (WAT) of genetically obese (ob/ob) mice (see FIG. 4B), supporting our data that Tec kinase is a modulator of adipogenesis. Expression profiling studies confirm the particular relevance of PTPRB as regulator of enery metabolism in mammals. PTPRB is rather ubiquitously expressed, with highest expression levels in lung tissue (see FIG. 9A).

Further, we show that the mammalian homologue of the Drosophilia PTPRB gene, is regulated by fasting. In this invention, we used mouse models of insulin resistance and/or diabetes, such as mice carrying gene knockouts in the leptin pathway (for example, ob (leptin) or db (leptin receptor) mice) to study the expression of the protein of the invention. Such mice develop typical symptoms of diabetes, show hepatic lipid accumulation and frequently have increased plasma lipid levels (see Bruning et al, 1998, Mol. Cell. 2:449-569). We found, for example, that the expression of PTPRB is strongly downregulated in the metabolically active tissue (for example, WAT) of fasted mice (see FIG. 9B).

1. A pharmaceutical composition comprising a nucleic acid molecule of the protein tyrosine phosphatase PTP10D, non-receptor protein tyrosine kinase Tec, or egg derived tyrosine phosphatase (EDTP) gene family or a polypeptide encoded thereby or a fragment or a variant of said nucleic acid molecule or said polypeptide or an antibody, an aptamer or another receptor recognizing a nucleic acid molecule of the PTP10D, non-receptor protein tyrosine kinase Tec, or EDTP gene family or a polypeptide encoded thereby together with pharmaceutically acceptable carriers, diluents and/or adjuvants.

2. The composition of claim 1, wherein the nucleic acid molecule is a vertebrate or insect PTP10D, Tec, or EDTP nucleic acid, particularly a nucleic acid encoding a Drosophila PTP10D (GadFly Accession Number CG 1817), human PTP10D homologous protein (PTPRB, Genbank Accession Number XM 006789.7 for the cDNA (SEQ ID NO: 3), Genbank Accession Number XP 006789.4 for the protein (SEQ ID NO: 4), and mouse receptor-type protein tyrosine phosphatase (Genbank Accession Number AF157628 for the cDNA, Genbank Accession Number AAF80346 for the protein), a Drosophila Btk29A (Gadfly Accession Numbers CG18355 and CG8049), human tec protein tyrosine kinase (Genbank Accession Number NM 003215.1 (formerly XM 044444.3) for the cDNA (SEQ ID NO: 1), Genbank Accession Number NP 003206.1 (formerly XP 044444) for the protein (SEQ ID NO: 2), and mouse protein tyrosine kinase TecIV (Genbank Accession Number AAD43402.1), or a Drosophila EDTP (Gadfly Accession Number CG6542), or a human EDTP homologous proteins on chromosome 3 (Genbank Accession Numbers XM 056609.3 and NM 022485.2 for the cDNAs, XP 056609 and NP 071930 for the proteins), cDNA of SEQ ID NO: 5 encoding protein of SEQ ID NO: 6, predicted cDNA from Genbank Accession Numbers, XM 056748, and BC 001674, further human proteins with Genbank Accession Numbers AAH35690.1, AAL55787, BAC11211, and CAD28494), or a fragment thereof or a variant thereof.

3. The composition of claim 1, wherein said nucleic acid molecule
(a) hybridizes at 50° C. in a solution containing 1×SSC and 0.1 SDS to a nucleic acid molecule as defined in claim 2 or a nucleic acid molecule which is complementary thereto;
(b) it is degenerate with respect to the nucleic acid molecule of (a)
(c) encodes a polypeptide which is at least 85%, preferably at least 90%, more preferably at least 95%, more preferably at least 98% and up to 99.6% identical to PTP10D, Tec, or EDTP polypeptides as defined in claim 2;
(d) differs from the nucleic acid molecule of (a) to (c) by mutation and wherein said mutation causes an alteration, deletion, duplication or premature stop in the encoded polypeptide.

4. The composition of claim 1, wherein the nucleic acid molecule is a DNA molecule, particularly a cDNA or a genomic DNA.

5. The composition of claim 1, wherein said nucleic acid encodes a polypeptide contributing to regulating the energy homeostasis and/or the metabolism of triglycerides.

6. The composition of claim 1, wherein said nucleic acid molecule is a recombinant nucleic acid molecule.

7. The composition of claim 1, wherein the nucleic acid molecule is a vector, particularly an expression vector.

8. The composition of claim 1, wherein the polypeptide is a recombinant polypeptide.

9. The composition of claim 8, wherein said recombinant polypeptide is a fusion polypeptide.

10. The composition of claim 1, wherein said-nucleic acid molecule is selected from hybridization probes, primers and anti-sense oligonucleotides.

11. The composition of claim 1 which is a diagnostic composition.

12. The composition of claim 1 which is a therapeutic composition.

13. The composition of claim 1 for the manufacture of an agent for detecting and/or verifying, for the treatment, alleviation and/or prevention of an disorders, including metabolic diseases such as obesity and other body-weight regulation disorders as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea and others, in cells, cell masses, organs and/or subjects.

14. Use of a nucleic acid molecule of the PTP10D, Tec, or EDTP gene family or use of a polypeptide encoded thereby or use of a fragment or a variant of said nucleic acid molecule or said polypeptide or use of an antibody, an aptamer or another receptor recognizing said nucleic acid molecule or said polypeptide encoded thereby for controlling the function of a gene and/or a gene product which is influenced and/or modified by a PTP10D, Tec, or EDTP homologous polypeptide.

15. Use of the nucleic acid molecule of the PTP10D, Tec, or EDTP gene family or use of a polypeptide encoded thereby or use of a fragment or a variant of said nucleic acid molecule or use of said polypeptide or an antibody, an aptamer or another receptor recognizing said nucleic acid molecule or said polypeptide encoded thereby or use of an effector said nucleic acid molecule or said polypeptide encoded thereby for identifying substances capable of interacting with a PTP10D, Tec, or EDTP homologous polypeptide.

16. A non-human transgenic animal exhibiting a modified expression of a PTP10D, Tec, or EDTP homologous polypeptide.

17. The animal of claim 16, wherein the expression of the PTP10D, Tec, or EDTP homologous polypeptide is increased and/or reduced.

18. A recombinant host cell exhibiting a modified expression of a PTP10D, Tec, or EDTP homologous polypeptide or a recombinant host cell which comprises a nucleic acid molecule as defined in claim 1.

19. The cell of claim 18 which is a human cell.

20. A method of identifying a (poly)peptide involved in the regulation of energy homeostasis and/or metabolism of triglycerides in a mammal comprising the steps of
(a) contacting a collection of (poly)peptides with a PTP10D, Tec, or EDTP homologous polypeptide or a fragment thereof under conditions that allow binding of said (poly)peptides;
(b) removing (poly)peptides which do not bind and (c) identifying (poly)peptides that bind to said PTP10D, Tec, or EDTP homologous polypeptide.

21. A method of screening for an agent which modulates the interaction of a PTP10D, Tec, or EDTP homologous polypeptide with a binding target/agent, comprising the steps of
(a) incubating a mixture comprising
(aa) a PTP10D, Tec, or EDTP homologous polypeptide, or a fragment thereof;
(ab) a binding target/agent of said PTP10D, Tec, or EDTP homologous polypeptide or fragment thereof; and
(ac) a candidate agent
under conditions whereby said PTP10D, Tec, or EDTP polypeptide or fragment thereof specifically binds to said binding target/agent at a reference affinity;
b) detecting the binding affinity of said PTP10D, Tec, or EDTP polypeptide or fragment thereof to said binding target to determine an (candidate) agent-biased affinity; and
(c) determining a difference between (candidate) agent-biased affinity and the reference affinity.

22. A method of screening for an agent which modulates the activity of a PTP10D, Tec, or EDTP homologous polypeptide comprising the steps of
(a) incubating a mixture comprising
(aa) a PTP10D, Tec, or EDTP homologous polypeptide, or a fragment thereof and
(ab) a candidate agent
under conditions whereby said PTP10D, Tec, or EDTP polypeptide or fragment thereof exhibits a reference activity,
(b) detecting the activity of said PTP10D, Tec, or EDTP polypeptide or fragment thereof to determine an (candidate) agent-biased activity and
(c) determining a difference between (candidate) agent-biased activity and reference activity.

23. The method of claim 22, wherein the activity is a kinase or phosphatase activity.

24. A method of producing a composition comprising mixing the (poly)peptide identified by the method of claim 20 with a pharmaceutically acceptable carrier, diluent and/or adjuvant.

25. The method of claim 24 wherein said composition is a pharmaceutical composition for preventing, alleviating or treating of diseases and disorders, including metabolic diseases such as obesity and other body-weight regulation disorders as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea and other diseases and disorders.

26. Use of a (poly)peptide as identified by the method of claim 20 for the preparation of a pharmaceutical composition for the treatment, alleviation and/or prevention of diseases and disorders, including metabolic diseases such as obesity and other body-weight regulation disorders as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea and other diseases and disorders.

27. Use of a nucleic acid molecule of the PTP10D, Tec, or EDTP family or of a fragment thereof for the preparation of a non-human animal which over- or under-expresses the PTP10D, Tec, or EDTP gene product.

28. Kit comprising at least one of
(a) a PTP10D, Tec, or EDTP nucleic acid molecule or a fragment thereof;
(b) a vector comprising the nucleic acid of (a);
(c) a host cell comprising the nucleic acid of (a) or the vector of (b);
(d) a polypeptide encoded by the nucleic acid of (a);
(e) a fusion polypeptide encoded by the nucleic acid of (a);
(f) an antibody, an aptamer or another receptor against the nucleic acid of (a) or the polypeptide of (d) or (e) and
(g) an anti-sense oligonucleotide of the nucleic acid of (a).


SEQUENCE LISTING (20 sequences)

SEQ ID NO: 1 - DNA, 3650 bp, Homo sapiens
cggcggccgcggatcccggcggcgatccgacctcgcagtctccccaggtccgccagcagccggttcagccagaatactgggatcttcagtggcaggaggagtaatcagaagacggagatgaattttaacactattttggaggagattcttattaagaggtcacagcagaaaaagaagacatcgcccttaaactacaaagagagactttttgtacttacaaagtccatgctaacctactatgagggtcgagcagagaagaaatacagaaaggggtttattgatgtttcaaaaatcaagtgtgtggaaatagtgaagaatgatgatggtgtcattccctgtcaaaataagtatccatttcaggttgttcatgatgctaacacactttacatttttgcacctagtccacaaagcagggacctgtgggtgaagaagttaaaagaagaaataaagaacaacaataatattatgattaaatatcatcctaaattctggacagatggaagttatcagtgttgtagacaaactgaaaaattagcacccggatgtgaaaaatacaatctttttgagagcagtataagaaaagcactacctccagcaccagaaacaaagaagcgaaggcctcccccaccaattccactagaagaagaagataatagtgaagaaatcgttgtagccatgtatgatttccaagcagcagaaggacatgatctcagattagagagaggccaagagtatctcattttagaaaagaatgatgtgcattggtggagagcaagagataaatatgggaatgaaggatatatcccaagtaattacgtaacgggaaagaaatcaaacaacttagatcaatatgaatggtattgcagaaatatgaatagaagcaaggcagagcaactcctccgcagtgaagataaagaaggtggttttatggtaagggattccagtcaaccaggcttgtacacagtctccctttataccaagtttggaggagaaggttcatcgggttttaggcattatcatataaaggaaacaacaacatctccaaagaagtattacctagctgaaaaacatgcttttggctccattcctgagattattgaatatcataagcacaatgcagcaggacttgtcaccaggcttcggtacccagttagtgtgaaagggaagaatgcacccaccactgcaggattcagctatgagaaatgggagattaacccttcagaactgacctttatgagggaattgggaagtggactgtttggagtggtgaggcttggcaaatggcgagcccagtacaaagtcgcaatcaaagctattcgggaaggtgcaatgtgcgaggaggactttatagaagaagctaaagtgatgatgaagctgacacacccgaagttagtgcagctttatggtgtgtgcacccagcagaaaccaatatacattgttactgagttcatggaaaggggctgccttctgaatttcctccgacagagacaaggtcatttcagtagagacgtactgctgagcatgtgtcaggatgtgtgtgaagggatggagtatctggagagaaacagcttcatccacagagatctggctgccagaaattgtctagtaagtgaggcgggagttgtaaaagtatctgattttggaatggccaggtattttctggatgatcagtacacaagttcttctggtgctaagtttcctgtgaagtggtgtccacctgaagtgtttaattacagccgcttcagcagcaaatcagatgtctggtcatttggtgttttaatgtgggaagtattcacggaaggcagaatgccttttgaaaaatacaccaattatgaagtggtaaccatggttactcgaggccaccgactctaccagccgaagttggcgtccaactatgtgtatgaggtgatgctgagatgttggcaggagaaaccagagggaaggccttctttcgaagatctgctgcgcacaatagatgaactagttgaatgtgaagaaacttttggaagataagtgatgtgtgaccagtggctcccagattcccaagcacaaggaaggatgggcattttgtggcttttaatttattgagcacttggacatgtagatcattttacttatacagtggaaacacataaataatttgcttctagaccagcctctgtctagacttgcttctagacagaatctcccagagtgtggaaatgttgccttagaaatggtgattaaaatcactcatttctattcattcctcaggcacttgagtgacagttgtttaccaggcactgtgtgtagccccagggtttggccattcaggggtgcacacatgggaccatgttagctgatgccagttgaaggccagggtatttgggaaggggaagggtattagagtcatgaccaagcaacccttctttttccctttgacttctacagaaatctgggcctgagacattgtctacaattgggttctagatacatcaggaacccatcttggataaataaatacctatcttttgttttgaaaacatctcagttttcaagactgctcttagtattacatgaacaatatttgtatgctgtatatattgtaaatatatataatatataaagttatatatttatgagaaacacgaattgtcttttaattgaaacttttaatcctgtagtataggagttcaccttcttaggactagagactgtgccttatagctgttaattcatttccccctgaacatcaaatatgcctgaagagaagaaagtctagattcttctatgagtaacgccccctcctcactcaggtaaatgtgtctggggatgcctgtccagcttaaccacgtgcatttggcctatgtaatcctgcccatggtggccgcagctaatcagaatcagatggaaaattaaaccgggtaatctacttctaagccttaagaatattccctgggacacagacactataattggaagtgctgagctctggggcagaaggatcaggtgaccttcgcaacaaagtttgcccccacctcacataggacccggaagcagcctgagctgtggcggaggatccaggaagctacggagagaagcagccagcatggtgttccgtgcctcccggacgtttttcaggaggcctggttggacttgggttcctggatggtgggattgttgtacagcctctcaggagaccctgctgtcaagactgtgtgtgtggatttcccacccttagaagctctactaagacatcaacggaattagggccttcctttttgccttgtgagcgccaaggaaaagaaactatctcggtcacgtgagcgccacgaaagaaactgtatcagtcatccagagaccgtttattgcccaacacgttattcttgctgttggtggggtaactagccgaggaagacacagcgccttcccttcaggagttgcgtctcctctgcaggccacgatggtctgctctggagcattgggtgaacacacaggctggctgctctgggcagcgccttcactctgaccctggagaaccatttcatttcatcctggtcagtctagagtctgtgcaccaggcagtccatccactgaaggctgtgtttattcttttcctgtgcccctcataatggaagaaagtaaactgcttatcccgagccttaaaaaaa

SEQ ID NO: 2 - protein, 631 aa, Homo sapiens
MNFNTILEEILIKRSQQKKKTSPLNYKERLFVLTKSMLTYYEGRAEKKYRKGFIDVSKIKCVEIVKNDDGVIPCQNKYPFQVVHDANTLYIFAPSPQSRDLWVKKLKEEIKNNNNIMIKYHPKFWTDGSYQCCRQTEKLAPGCEKYNLFESSIRKALPPAPETKKRRPPPPIPLEEEDNSEEIVVAMYDFQAAEGHDLRLERGQEYLILELNDVHWWRARDKYGNEGYIPSNYVTGKKSNNLDQYEWYCRNMNRSKAEQLLRSEDKEGGFMVRDSSQPGLYTVSLYTKFGGEGSSGFRHYHIKETTTSPKKYYLAEKHAFGSIPEIIEYHKHNAAGLVTRLRYPVSVKGKNAPTTAGFSYEKWEINPSELTFMRELGSGLFGVVRLGLWRAQYKVAIKAIREGAMCEEDFIEEAKVMMKLTHPKLVQLYGVCTQQKPIYIVTEFMERGCLLNFLRQRQGHFSRDVLLSMCQDVCEGMEYLERNSFIHRDKAARNCLVSEAGVVKVSDFGMARYFLDDQYTSSSGAKFPVKWCPPEVFNYSRFSSKSDVWSFGVLMWEVFTEGRMPFEKYTNYEVVTMVTRGHRLYQPKLASNYVYEVMLRCWQEKPEGRPSFEDLLRTIDELVECEETFGR

SEQ ID NO: 3 - DNA, 6329 bp, Homo sapiens
gtctcctctggatcttaactactgagcgcaatgctgagccatggagccgggttggccttgtggatcacactgagcctgctgcagactggactggcggagccagagagatgtaacttcaccctggcggagtccaaggcctccagccattctgtgtctatccagtggagaattttgggctcaccctgtaactttagcctcatctatagcagtgacaccctgggggccgcgttgtgccctacctttcggatagacaacaccacatacggatgtaaccttcaagatttacaagcaggaaccatctataacttcaggattatttctctggatgaagagagaacagtggtcttgcaaacagatcctttacctcctgctaggtttggagtcagtaaagagaagacgacttcaaccagcttgcatgtttggtggactccttcttccggaaaagtcacctcatatgaggtgcaattatttgatgaaaataaccaaaagatacagggggttcaaattcaagaaagtacttcatggaatgaatacacttttttcaatctcactgctggtagtaaatacaatattgccatcacagctgtttctggaggaaaacgttctttttcagtttataccaatggatcaacagtgccatctccagtgaaagatattggtatttccacaaaagccaattctctcctgatttcctggtcccatggttctgggaatgtggaacgataccggctgatgctaatggataaagggatcctagttcatggcggtgttgtggacaaacatgctacttcctatgcttttcacgggctgacccctggctacctctacaacctcactgttatgactgaggctgcagggctgcaaaactacaggtggaaactagtcaggacagcccccatggaagtctcaaatctgaaggtgacaaatgatggcagtttgacctctctaaaagtcaaatggcaaagacctcctggaaatgtggattcttacaatatcaccctgtctcacaaagggaccatcaaggaatccagagtattagcaccttggattactgaaactcactttaaagagttagtccccggtcgactttatcaagttactgtcagctgtgtctctggtgaactgtctgctcagaagatggcagtgggcagaacatttccagacaaagttgcaaacctggaggcaaacaataatggcaggatgaggtctcttgtagtgagctggtcgccccctgctggagactgggagcagtatcggatcctactcttcaatgattctgtggtgctgctcaacatcactgtgggaaaggaagaaacacagtatgtcatggatgacacggggctcgtaccgggaagacagtatgaggtggaagtcattgttgagagtggaaatttgaagaattctgagcgttgccaaggcaggacagtccccctggctgtcctccagcttcgtgtcaaacatgccaatgaaacctcactgagtatcatgtggcagacccctgtagcagaatgggagaaatacatcatttccctagctgacagagacctcttactgatccacaagtcactctccaaagatgccaaagaattcacttttactgacctggtgcctggacgaaaatacatggctacagtcaccagtattagtggagacttaaaaaattcctcttcagtaaaaggaagaacagtgcctgcccaagtgactgacttgcatgtggccaaccaaggaatgaccagtagtctgtttactaactggacccaggcacaaggagacgtagaattttaccaagtcttactgatccatgaaaatgtggtcattaaaaatgaaagcatctccagtgagaccagcagatacagcttccactctctcaagtccggcagcctgtactccgtggtggtaacaacagtgagtggagggatctcttcccgacaagtggttgtggagggaagaacagtcccttccagtgtgagtggagtaacggtgaacaattccggtcgtaatgactacctcagcgtttcctggctgctggcgcccggagatgtggataactatgaggtaacattgtctcatgacggcaaggtggttcagtcccttgtcattgccaagtctgtcagagaatgttccttcagctccctcaccccaggccgcctctacaccgtgaccataactacaaggagtggcaagtatgaaaatcactccttcagccaagagcggacagtgcctgacaaagtccagggagtcagtgttagcaactcagccaggagtgactatttaagggtatcctgggtgcatgccactggagactttgatcactatgaagtcaccattaaaaacaaaaacaacttcattcaaactaaaagcattcccaagtcagaaaacgaatgtgtatttgttcagctagtccctggacggttgtacagtgtcactgttactacaaaaagtggacaatatgaagccaatgaacaagggaatgggagaacaattccagagcctgttaaggatctaacattgcgcaacaggagcactgaggacttgcatgtgacttggtcaggagctaatggggatgtcgaccaatatgagatccagctgctcttcaatgacatgaaagtatttcctccttttcaccttgtaaataccgcaaccgagtatcgatttacttccctaacaccaggccgccaatacaaaattcttgtcttgacgattagcggggatgtacagcagtcagccttcattgagggcttcacagttcctagtgctgtcaaaaatattcacatttctcccaatggagcaacagatagcctgacggtgaactggactcctggtgggggagacgttgattcctacacggtgtcggcattcaggcacagtcaaaaggttgactctcagactattcccaagcacgtctttgagcacacgttccacagactggaggccggggagcagtaccagatcatgattgcctcagtcagcgggtccctgaagaatcagataaatgtggttgggcggacagttccagcatctgtccaaggagtaattgcagacaatgcatacagcagttattccttaatagtaagttggcaaaaagctgctggtgtggcagaaagatatgatatcctgcttctaactgaaaatggaatccttctgcgcaacacatcagagccagccaccactaagcaacacaaatttgaagatctaacaccaggcaagaaatacaagatacagatcctaactgtcagtggaggcctctttagcaaggaagcccagactgaaggccgaacagtcccagcagctgtcaccgacctgaggatcacagagaactccaccaggcacctgtccttccgctggaccgcctcagagggggagctcagctggtacaacatctttttgtacaacccagatgggaatctccaggagagagctcaagttgacccactagtccagagcttctctttccagaacttgctacaaggcagaatgtacaagatggtgattgtaactcacagtggggagctgtctaatgagtctttcatatttggtagaacagtcccagcctctgtgagtcatctcagggggtccaatcggaacacgacagacagcctttggttcaactggagtccagcctctggggactttgacttttatgagctgattctctataatcccaatggcacaaagaaggaaaactggaaagacaaggacctgacggagtggcggtttcaaggccttgttcctggaaggaagtacgtgctgtgggtggtaactcacagtggagatctcagcaataaagtcacagcggagagcagaacagctccaagtcctcccagtcttatgtcatttgctgacattgcaaacacatccttggccatcacgtggaaagggcccccagactggacagactacaacgactttgagctgcagtggttgcccagagatgcacttactgtcttcaacccctacaacaacagaaaatcagaaggacgcattgtgtatggtcttcgtccagggagatcctatcaattcaacgtcaagactgtcagtggtgattcctggaaaacttacagcaaaccaatttttggatctgtgaggacaaagcctgacaagatacaaaacctgcattgccggcctcagaactccacggccattgcctgttcttggatccctcctgattctgactttgatggttatagtattgaatgccggaaaatggacacccaagaagttgagttttccagaaagctggagaaagaaaaatctctgctcaacatcatgatgctagtgccccataagaggtacctggtgtccatcaaagtgcagtcggccggcatgaccagcgaggtggttgaagacagcactatcacaatgatagaccgcccccctcctccacccccacacattcgtgtgaatgaaaaggatgtgctaattagcaagtcttccatcaactttactgtcaactgcagctggttcagcgacaccaatggagctgtgaaatacttcacagtggtggtgagagaggctgatggcagtgatgagctgaagccagaacagcagcaccctctcccttcctacctggagtacaggcacaatgcctccattcgggtgtatcagactaattattttgccagcaaatgtgccgaaaatcctaacagcaactccaagagttttaacattaagcttggagcagagatggagagcctaggtggaaaatgcgatcccactcagcaaaaattctgtgatggaccactgaagccacacactgcctacagaatcagcattcgagcttttacacagctctttgatgaggacctgaaggaattcacaaagccactctattcagacacatttttttctttacccatcactactgaatcagagcccttgtttggagctattgaaggtgtgagtgctggtctgtttttaattggcatgctagtggctgttgttgccttattgatctgcagacagaaagtgagccatggtcgagaaagaccctctgcccgtctgagcattcgtagggatcgaccattatctgtccacttaaacctgggccagaaaggtaaccggaaaacttcttgtccaataaaaataaatcagtttgaagggcatttcatgaagctacaggctgactccaactaccttctatccaaggaatacgaggagttaaaagacgtgggccgaaaccagtcatgtgacattgcactcttgccggagaatagagggaaaaatcgatacaacaatatattgccctatgatgccacgcgagtgaagctctccaatgtagatgatgatccttgctctgactacatcaatgccagctacatccctggcaacaacttcagaagagaatacattgtcactcagggaccgcttcctggcaccaaggatgacttctggaaaatggtgtgggaacaaaacgttcacaacatcgtcatggtgacccagtgtgttgagaagggccgagtaaagtgtgatcattactggccagcggaccaggattccctctactatggggacctcatcctgcagatgctctcagagtccgtcctgcctgagtggaccatccgggagtttaagatatgcggtgaggaacagcttgatgcacacagactcatccgccactttcactatacggtgtggccagaccatggagtcccagaaaccacccagtctctgatccagtttgtgagaactgtcagggactacatcaacagaagcccgggtgctgggcccactgtggtgcactgcagtgctggtgtgggtaggactggaacctttattgcattggaccgaatcctccagcagttagactccaaagactctgtggacatttatggagcagtgcacgacctaagacttcacagggttcacatggtccagactgagtgtcagtatgtctacctacatcagtgtgtaagagatgtcctcagagcaagaaagctacggagtgaacaagaaaaccccttgtttccaatctatgaaaatgtgaatccagagtatcacagagatccagtctattcaaggcattgagaatgtacctgaagagctcctggataaaaattattcactgtgtgatttgtttttaaaaacttgcttcatgccctacagaggtgccagctatttctgttgatactatgtataatttattaatctggagaatgtttaaaattttatataatttaaaggtaacagatattattgtacatagttgtattttgtagtttcttctgtaaatatgtatttttcataatgtttaatattaagctttatataatactatttttccacactaaagtgttcatgacttgttctacataaaactaattcaacctgta

SEQ ID NO: 4 - protein, 1997 aa, Homo sapiens
MLSHGAGLALWITLSLLQTGLAEPERCNFTLAESKASSHSVSIQWRILGSPCNFSLIYSSDTLGAALCPTFRIDNTTYGCNLQDLQAGTIYNFRIISLDEERTVVLQTDPLPPARFGVSKEKTTSTSLHVWWTPSSGKVTSYEVQLFDENNQKIQGVQIQESTSWNEYTFFNLTAGSKYNIAITAVSGGKRSFSVYTNGSTVPSPVKDIGISTKANSLLISWSHGSGNVERYRLMLMDKGILVHGGVVDKHATSYAFHGLTPGYLYNLTVMTEAAGLQNYRWKLVRTAPMEVSNLKVTNDGSLTSLKVKWQRPPGNVDSYNITLSHKGTIKESRVLAPWITETHFKELVPGRLYQVTVSCVSGELSAQKMAVGRTFPDKVANLEANNNGRMRSLVVSWSPPAGDWEQYRILLFNDSVVLLNITVGKEETQYVMDDTGLVPGRQYEVEVIVESGNLKNSERCQGRTVPLAVLQLRVKHANETSLSIMWQTPVAEWEKYIISLADRDLLLIHKSLSKDAKEFTFTDLVPGRKYMATVTSISGDLKNSSSVKGRTVPAQVTDLHVANQGMTSSLFTNWTQAQGDVEFYQVLLIHENVVIKNESISSETSRYSFHSLKSGSLYSVVVTTVSGGISSRQVVVEGRTVPSSVSGVTVNNSGRNDYLSVSWLLAPGDVDNYEVTLSHDGKVVQSLVIAKSVRECSFSSLTPGRLYTVTITTRSGKYENHSFSQERTVPDKVQGVSVSNSARSDYLRVSWVHATGDFDHYEVTIKNKNNFIQTKSIPKSENECVFVQLVPGRLYSVTVTTKSGQYEANEQGNGRTIPEPVKDLTLRNRSTEDLHVTWSGANGDVDQYEIQLLFNDMKVFPPFHLVNTATEYRFTSLTPGRQYKILVLTISGDVQQSAFIEGFTVPSAVKNIHISPNGATDSLTVNWTPGGGDVDSYTVSAFRHSQKVDSQTIPKHVFEHTFHRLEAGEQYQIMIASVSGSLKNQINVVGRTVPASVQGVIADNAYSSYSLIVSWQKAAGVAERYDILLLTENGILLRNTSEPATTKQHKFEDLTPGKKYKIQILTVSGGLFSKEAQTEGRTVPAAVTDLRITENSTRHLSFRWTASEGELSWYNIFLYNPDGNLQERAQVDPLVQSFSFQNLLQGRMYKMVIVTHSGELSNESFIFGRTVPASVSHLRGSNRNTTDSLWFNWSPASGDFDFYELILYNPNGTKKENWKDKDLTEWRFQGLVPGRKYVLWVVTHSGDLSNKVTAESRTAPSPPSLMSFADIANTSLAITWKGPPDWTDYNDFELQWLPRDALTVFNPYNNRKSEGRIVYGLRPGRSYQFNVKTVSGDSWKTYSKPIFGSVRTKPDKIQNLHCRPQNSTAIACSWIPPDSDFDGYSIECRKMDTQEVEFSRKLEKEKSLLNIMMLVPHKRYLVSIKVQSAGMTSEVVEDSTITMIDRPPPPPPHIRVNEKDVLISKSSINFTVNCSWFSDTNGAVKYFTVVVREADGSDELKPEQQHPLPSYLEYRHNASIRVYQTNYFASKCAENPNSNSKSFNIKLGAEMESLGGKCDPTQQKFCDGPLKPHTAYRISIRAFTQLFDEDLKEFTKPLYSDTFFSLPITTESEPLFGAIEGVSAGLFLIGMLVAVVALLICRQKVSHGRERPSARLSIRRDRPLSVHLNLGQKGNRKTSCPIKINQFEGHFMKLQADSNYLLSKEYEELKDVGRNQSCDIALLPENRGKNRYNNILPYDATRVKLSNVDDDPCSDYINASYIPGNNFRREYIVTQGPLPGTKDDFWKMVWEQNVHNIVMVTQCVEKGRVKCDHYWPADQDSLYYGDLILQMLSESVLPEWTIREFKICGEEQLDAHRLIRHFHYTVWPDHGVPETTQSLIQFVRTVRDYINRSPGAGPTVVHCSAGVGRTGTFIALDRILQQLDSKDSVDIYGAVHDLRLHRVHMVQTECQYVYLHQCVRDVLRARKLRSEQENPLFPIYENVNPEYHRDPVYSRH

SEQ ID NO: 5 - DNA, 2152 bp, Homo sapiens
gaaggcggtggctgaggcggttccggaggttctagtgtcggagttgggtgcaggcaggtgccatgggcccgcttgaggcacactgaggggacgcggggctgggccatggccggcgctcgggccgccgccgccgctgcctcggcggggtcctcggcctcttcaggcaaccagccgcctcaggagctggggcttggggagctgctggaggagttctcccggactcagtaccgggccaaggatggcagcgggaccggcggctctaaggttgagcgcattgagaagagatgtctggagctgtttggccgagactactgtttcagcgtgattccaaacacgaatggggatatctgtggccactatccccggcacatcgtgttcctggagtatgagagttctgagaaggagaaagacacgtttgagagtaccgtacaggtgagcaagttgcaagacctcatccaccgcagcaagatggcccggtgcagaggacggtttgtctgcccagtaatcctgttcaagggcaagcacatttgcaggtcggccacactggctggatggggagagctgtatggacgctcaggctacaactattttttctcagggggtgcagatgatgcctgggcagatgtggaggacgtcacggaggaggactgtgctcttcgaagtggtgacacgcatctttttgataaggtcagaggctatgacatcaagctgcttcgatacctgtcagtcaaatacatctgtgacctgatggtggagaacaagaaggtgaagtttggcatgaatgtaacctcctctgagaaggtggacaaagcccagcgctatgccgacttcactctcctctccatcccgtatccaggctgtgaatttttcaaggaatataaagatcgggattacatggcagaagggctcatatttaactggaagcaggactacgttgatgccccattgagcatccccgacttcctgactcactctctgaacattgactggagccagtatcagtgttgggatctggtgcaacaaacacaaaactacctgaagctgctgctttccttagttaacagtgatgatgacagcgggctgctggtacactgtatctcaggctgggatcggacccccctcttcatctccctcctgcgcctttccttgtgggctgatgggctcatccacacgtccctgaagcccactgagatcctctacctcactgtggcctatgactggttcctcttcgggcacatgttggtagatcggctcagcaaaggggaggagattttcttcttctgcttcaattttttgaagcatattacctccgaggagttctctgctctgaagacccagaggaggaagagtttgccagcccgggatggaggcttcaccctggaagacatctgcatgctgagacgaaaggaccgtggcagcaccaccagccttggcagcgacttctccctggtcatggagagttccccaggagccactgggagcttcacctatgaggccgtggagctggtcccagcaggagcgccaactcaggcagcttggcttgcagccctgagtgatcgagagactcggctgcaggaggtgcgctcagccttcttggctgcgtacagcagcacagtggggcttcgggcagtagcccccagtccttccggtgccatcgggggcctgctggagcaatttgcccgtggtgttggactccggagcatcagcagcaatgccttgtgaagaagccagcccatgacattttcctgctcctctctcagctgagcccttagcagagaatcaaagccatgcctggccgaaggggtacttccaggtcaggggaaatttcagtcccccatctccatcatgaacatggcagccccaaagctgagcaaggccaaagacagggttttccaacccccagcctcttgactggtgaccaccaccccttcttgtcactgtctcccacccaccccatctttgctgggattcccatcaactctcagaactgtgtggggtttccctggggccttgtggaagccatgacttcacaaagaccctacctgtcagttcttgtttctggggaggagggatcacctgcactgagaatgaggcagtttgacacagatcacaaaataaaatcaaagtctttttgaaaaaaaaaaaaaaaaaa

SEQ ID NO: 6 - protein, 538 aa, Homo sapiens
MAGARAAAAAASAGSSASSGNQPPQELGLGELLEEFSRTQYRAKDGSGTGGSKVERIEKRCLELFGRDYCFSVIPNTNGDICGHYPRHIVFLEYESSEKEKDTFESTVQVSKLQDLIHRSKMARCRGRFVCPVILFKGKHICRSATLAGWGELYGRSGYNYFFSGGADDAWADVEDVTEEDCALRSGDTHLFDKVRGYDIKLLRYLSVKYICDLMVENKKVKFGMNVTSSEKVDKAQRYADFTLLSIPYPGCEFFKEYKDRDYMAEGLIFNWKQDYVDAPLSIPDFLTHSLNIDWSQYQCWDLVQQTQNYLKLLLSLVNSDDDSGLLVHCISGWDRTPLFISLLRLSLWADGLIHTSLKPTEILYLTVAYDWFLFGHMLVDRLSKGEEIFFFCFNFLKHITSEEFSALKTQRRKSLPARDGGFTLEDICMLRRKDRGSTTSLGSDFSLVMESSPGATGSFTYEAVELVPAGAPTQAAWLAALSDRETRLQEVRSAFLAAYSSTVGLRAVAPSPSGAIGGLLEQFARGVGLRSISSNAL

SEQ ID NO: 7 - DNA, 19 bp, Artificial Sequence
  mouse Tec forward primer
tcttggcttgtctcggcac

SEQ ID NO: 8 - DNA, 23 bp, Artificial Sequence
  mouse Tec reverse primer
ggacatcctctgtgtcctcagtg

SEQ ID NO: 9 - DNA, 22 bp, Artificial Sequence
  mouse Tec Taqman probe
cgctccgtcaaggtgtcggacg

SEQ ID NO: 10 - DNA, 22 bp, Artificial Sequence
  mouse PTPRB forward primer
gagaatacatcgccactcaggg

SEQ ID NO: 11 - DNA, 18 bp, Artificial Sequence
  mouse PTPRB reverse primer
ctcccacgccatcttcca

SEQ ID NO: 12 - DNA, 25 bp, Artificial Sequence
  mouse PTPRB Taqman probe
cgcttccaggcaccaaggatgactt

SEQ ID NO: 13 - protein, 1447 aa, Drosophila
  misc_feature (365)..(378): Xaa can be any naturally occurring amino acid
EIQFSRALPGTKYNFWLYYTNFTHHDWLTWTVTITTAPDPPSNLSVQVRSGKNAIILWSPPTQGSYTAFKIKVLGLSEASSSYNRTFQVNDNTFQHSVKELTPGATYQVQAYTIYDGKESVAYTSRNFTTKPNTPGKFIVWFRNETTLLVLWQPPYPAGIYTHYKVSIEPPDANDSVLYVEKEGEPPGPAQAAFKGLVPGRAYNISVQTMSEDEISLPTTAQYRTVPLRPLNVTFDRDFITSNSFRVLWEAPKGISEFDKYQVSVATTRRQSTVPRSNEPVAFFDFRDIAEPGKTFNVIVKTVSGKVTSWPATGDVTLRPLPVRNLRSINDDKTNTMIITWEADPASTQDEYRIVYHELETFNGXXXXXXXXXXXXXXESLLPGRNYSLSVQAVSKKMESNETSIFVVTRPSSPIIEDLKSIRMGLNISWKSDVNSKQEQYEVLYSRNGTSDLRTQKTKESRLVIKNLQPGAGYELKVFAVSHDLRSEPHAYFQAVYPNPPRNMTIETVRSNSVLVHWSPPESGEFTEYSIRYRTDSEQQWVRLPSVRSTEADITDMTKGEKYTIQVNTVSFGVESPVPQEVNTTVPPNPVSNIIQLVDSRNITLEWPKPEGRVESYILKWWPSDNPGRVQTKNVSENKSADDLSTVRVLIGELMPGVQYKFDIQTTSYGILSGITSLYPRTMPLIQSDVVVANGEKEDERDTITLSYTPTPQSSSKFDIYRFSLGDAEIRDKEKLANDTDRKVTFTGLVPGRLYNITVWTVSGGVASLPIQRQDRLYPEPITQLHATNITDTEISLRWDLPKGEYNDFDIAYLTADNLLAQNMTTRNEITISDLRPHRNYTFTVVVRSGTEXXXXXXXXXXXXXFTTNEAVPGRVERFHPTDVQPSEINFEWSLPSSEANGVIRQFSIAYTNINNLTDAGMQDFESEEAFGVIKNLKPGETYVFKIQAKTAIGFGPEREYRQTMPILAPPRPATQVVPTEVYRSSSTIQIRFRKNYFSDQNGQVRMYTIIVAEDDAKNASGLEMPSWLDVQSYSVWLPYQAIDPYYPFENRSVEDFTIGTENCDNHKIGYCNGPLKSGTTYRVKVRAFTGADKFTDTAYSFPIQTDQDNTSXXXXXXXXXXXXXXXXXXXXFYXXXXXXXXXXXXDSRANDNMSLPDSVIEQNRPILIKNFAEHYRLMSAXXXXXXXXXXXXLKHVGRDQPCTFADLPCNRPKNRFTNILPYDHSRFKLQPVDDDEGSDYINANYVPGHNSPREFIVTQGPLHSTRDDFWRMCWESNSRAIVMLTRCFEKGREKCDQYWPNDTVPVFYGDIKVQILNDSHYADWVMTEFMLCRGSEQRILRHFHFTTWPDFGVPNPPQTLVRFVRAFRDRIGAEQRPIVVHCSAGVGRSGTFITLDRILQQINTSDYVDIFGIVYAMRKERVWMVQTEQQYICIHQCLLAVLEGKE

SEQ ID NO: 14 - protein, 1450 aa, Homo sapiens
EFTFTDLVPGRKYMATVTSISGDLKNSSSVKGRTVPAQVTDLHVANQGMTSSLFTNWTQAQGDVEFYQVLLIHENVVIKNESISSETSRYSFHSLKSGSLYSVVVTTVSGGISSRQVVVEGRTVPSSVSGVTVNNSGRNDYLSVSWLLAPGDVDNYEVTLSHDGKVVQSLVIAKSVRECSFSSLTPGRLYTVTITTRSGKYENHSFSQERTVPDKVQGVSVSNSARSDYLRVSWVHATGDFDHYEVTIKNKNNFIQTKSIPKSENECVFVQLVPGRLYSVTVTTKSGQYEANEQGNGRTIPEPVKDLTLRNRSTEDLHVTWSGANGDVDQYEIQLLFNDMKVFPPFHLVNTATEYRFTSLTPGRQYKILVLTISGDVQQSAFIEGFTVPSAVKNIHISPNGATDSLTVNWTPGGGDVDSYTVSAFRHSQKVDSQTIPKHVFEHTFHRLEAGEQYQIMIASVSGSLKNQINVVGRTVPASVQGVIADNAYSSYSLIVSWQKAAGVAERYDILLLTENGILLRNTSEPATTKQHKFEDLTPGKKYKIQILTVSGGLFSKEAQTEGRTVPAAVTDLRITENSTRHLSFRWTASEGELSWYNIFLYNPDGNLQERAQVDPLVQSFSFQNLLQGRMYKMVIVTHSGELSNESFIFGRTVPASVSHLRGSNRNTTDSLWFNWSPASGDFDFYELILYNPNGTKKENWKDKDLTEWRFQGLVPGRKYVLWVVTHSGDLSNKVTAESRTAPSPPSLMSFADIANTSLAITWKGPPDWTDYNDFELQWLPRDALTVFNPYNNRKSEGRIVYGLRPGRSYQFNVKTVSGDSWKTYSKPIFGSVRTKPDKIQNLHCRPQNSTAIACSWIPPDSDFDGYSIECRKMDTQEVEFSRKLEKEKSLLNIMMLVPHKRYLVSIKVQSAGMTSEVVEDSTITMIDRPPPPPPHIRVNEKDVLISKSSINFTVNCSWFSDTNGAVKYFTVVVREADGSDELKPEQQHPLPSYLEYRHNASIRVYQTNYFASKCAENPNSNSKSFNIKLGAEMESLGGKCDPTQQKFCDGPLKPHTAYRISIRAFTQLFDEDLKEFTKPLYSDTFFSLPITTESEPLFGAIEGVSAGLFLIGMLVAVVALLICRQKVSHGRERPSARLSIRRDRPLSVHLNLGQKGNRKTSCPIKINQFEGHFMKLQADSNYLLSKEYEELKDVGRNQSCDIALLPENRGKNRYNNILPYDATRVKLSNVDDDPCSDYINASYIPGNNFRREYIVTQGPLPGTKDDFWKMVWEQNVHNIVMVTQCVEKGRVKCDHYWPADQDSLYYGDLILQMLSESVLPEWTIREFKICGEEQLDAHRLIRHFHYTVWPDHGVPETTQSLIQFVRTVRDYINRSPGAGPTVVHCSAGVGRTGTFIALDRILQQLDSKDSVDIYGAVHDLRLHRVHMVQTECQYVYLHQCVRDVLRARK

SEQ ID NO: 15 - protein, 747 aa, Drosophila
MCSLTPNHMVNVTQQHLHDLLETFEKKSFEAAAFEEGTAEYDISKKCEYLFKLDYSLIELDNTNGLLSPRYPGRILIPEYEHGHMAKTLVPGNGLFGQVGGGVGGGGSSGTTATATPLNSSAGSTGSEGVGIQAFVTFANPLQTQQQHPLQQQYPSQQMHPLHAQYPSQQPHPLQQQQQQPSQQQPQNTIYEDQYDIQRMRELVTMAKYARCRQRFAVPVIMYRGKYICRSATLSVMPETYGRKVVDYAYDCLSGGNYTAPNGEENDADSTDESLITHMHDQAQSQFSYDEVIKSDIQLLHTLNVSTIVDLMVENRKIKYFMAVSSSEKADPNKHYKSFNLLSLPYPGCEFFKKFRDNNYMARNLHYNWKQTFNDANINIPNMGPAADIDVAWSEYRDWDLVAITQNYLRATLKYVQEENSGLLIHCISGWDRTPLFVSLVRLSLWADGLIHQSLNAMQMAYFTLAYDWYLFGHQLPDRLKRGEDIMFFCFHVLKFITDEEFSIVEHRKRTKTSSSSGSSVIVIKSDCCDDEPLKEDYILSFDQDSNDSYSNCSNCDMSITDNFYATTPAQVNPLTSRSPNPKRSRTSPISVPGSNARQRQESTSSNGSWQVVTDTGSIDSMMNGSYMMRFVAQQAADGGGSSNIPLCNGGNGYHCSINAASSGSGSGSGSSISNGSSTHGFANGSSKDVGGSTMASKQCINLRKQRLNAVRAIFIQAYGKTIGLKFKEGSSMNLATFIGNLADQLF

SEQ ID NO: 16 - protein, 417 aa, Homo sapiens
MARCRGRFVCPVILFKGKHICRSATLAGWGELYGRSGYNYFFSGGADDAWADVEDVTEEDCALRSGDTHLFDKVRGYDIKLLRYLSVKYICDLMVENKKVKFGMNVTSSEKVDKAQRYADFTLLSIPYPGCEFFKEYKDRDYMAEGLIFNWKQDYVDAPLSIPDFLTHSLNIDWSQYQCWDLVQQTQNYLKLLLSLVNSDDDSGLLVHCISGWDRTPLFISLLRLSLWADGLIHTSLKPTEILYLTVAYDWFLFGHMLVDRLSKGEEIFFFCFNFLKHITSEEFSALKTQRRKSLPARDGGFTLEDICMLRRKDRGSTTSLGSDFSLVMESSPGATGSFTYEAVELVPAGAPTQAAWLAALSDRETRLQEVRSAFLAAYSSTVGLRAVAPSPSGAIGGLLEQFARGVGLRSISSNAL

SEQ ID NO: 17 - protein, 275 aa, Homo sapiens [sequence omitted]

SEQ ID NO: 18 - protein, 650 aa, Homo sapiens
MAGARAAAAAASAGSSASSGNQPPQELGLGELLEEFSRTQYRAKDGSGTGGSKVERIEKRCLELFGRDYCFSVIPNTNGDICGHYPRHIVFLEYESSEKEKDTFESTVQVSKLQDLIHRSKMARCRGRFVCPVILFKGKHICRSATLAGWGELYGRSGYNYFFSGGADDAWADVEDVTEEDCALRSGDTHLFDKVRGYDIKLLRYLSVKYICDLMVENKKVKFGMNVTSSEKVDKAQRYADFTLLSIPYPGCEFFKEYKDRDYMAEGLIFNWKQDYVDAPLSIPDFLTHSLNIDWSQYQCWDLVQQTQNYLKLLLSLVNSDDDSGLLVHCISGWDRTPLFISLLRLSLWADGLIHTSLKPTEILYLTVAYDWFLFGHMLVDRLSKGEEIFFFCFNFLKHITSEEFSALKTQRRKSLPARDGGFTLEDICMLRRKDRGSTTSLGSDFSLVMESSPGATGSFTYEAVELVPAGAPTQAAWRKSHSSSPQSVLWNRPQPSEDRLPSQQGLAEARSSSSSSSNHSDNFFRMGSSPLEVPKPRSVDHPLPGSSLSTDYGSWQMVTGCGSIQERAVLHTDSSLPFSFPDELPNSCLLAALSDRETRLQEVRSAFLAAYSSTVGLRAVAPSPSGAIGGLLEQFARGVGLRSISSNAL

SEQ ID NO: 19 - protein, 650 aa, Homo sapiens [sequence omitted]

SEQ ID NO: 20 - protein, 521 aa, Homo sapiens [sequence omitted]